SEQUENCE LISTING (19 sequences)

SEQ ID NO: 1 - DNA, 4485 bp, Homo sapiens
gtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagctctattcaacaacaaattgactgacggctgtgcacactccatggctaagctccttgcatgcaggcagaacttcttggcattgaggctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaagttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaac

SEQ ID NO: 2 - protein, 1040 aa, Homo sapiens
MGEEGGSASHDEEERASVLLGHSPGCEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL

SEQ ID NO: 3 - DNA, 601 bp, Homo sapiens
cttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctcyggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctg

SEQ ID NO: 4 - DNA, 601 bp, Homo sapiens
ctcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggsgcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaat

SEQ ID NO: 5 - DNA, 330 bp, Homo sapiens
  misc_feature (142)..(142): n is a, c, g, or t
gactggctaactcctgcagtctctttaactggacagtttcaagaggaaaaccaagaatccttgaagctcaccattgtatcttcttttccaggttgtccaataactgcatcacctacctaggggcagaagccctcctgcaggncccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattc

SEQ ID NO: 6 - DNA, 25000 bp, Homo sapiens
atggcccttctcaaatcttctccatgccaagccccgggcctcaaatctagcaggttgttcttgtgcttttccaacagccctcatatatttgaatcaattatccccaacatcttcccacatattttcagcatttggtactgatccctctaccagatgccctgtccccaacaagcagcctcgcctcctatatctgcccaccaatggatgcgtcctcaaattctgtcctctctacatcaaacatgttttatgtcaccgacttcatcatcatcatctcacaaaacccagattctttcctcacagagcctatctgcattcgttctcattccaaggagaggtggcccttcccttcctaaattgcaagcccacccaccactgcccctggttcattcactttccctacttgggaaagcggctccatgaggttgactctgtccgttgcacatgtgaccactcttctccatcctgccggcttttcctcctcacttgaacacacttcttttaaaattatcttccctccaaagatgtttgtcgcagcattgtttgtaatttcaaaaaattaggacaagcacaaatgccaatcaatagaggattcattaagcaaaataccacatgcataaccggctacagaacacagccactgaaaaaaaagaacaagacagatctgcatatccaaaccagaatgctagctggaaaaaaaaaatgatgcagaatagcacgtatcatgtgaactcagttttcaaaaagaattctgtacgtaattatatgtcagtatatacataggaaaaatcttgaagaacatataccaaaccattaacaatggtcaactctgaggagtggaatttggagagctttcatctgatgtgttctacattcctttattgggatatttaggaccatgtaataacattatactcagaaaaattttatatacaaaagaaacttcccttaattctatcaatatcatcctctgtttccctttaattttcaccaacatccatccatccatccatccattcgtccccatccattttcaccaacatccatgtgtaagaccaggctggatggatacaacagcccttgggctcaaaacttgtacagtctggtgaatgtaacacggagccaccaaaatgtcatccagcagagaaagtggcaagtacgacagagctttccgttcagatccaagctttgctgctgaatttgggtaaatgactgagcctttatgagcctgtttcctcctctgaaaatggggattatagtaacaagcagagagaacaggagtggtgggtgggtagcgccttaagtaggtgttggtgcagaggactccatcccatctgggccacagaccatcaggaggcacgtggggacttggggacaaagaaaacaagcagcccatatctgccggcaggacctggcaaagtgggcgtgacaccaacaagttgcatcccagaggtgaccacaaagcgcccaggatttgcaaagctaaggtcaagtggaggcaggaggagacagtttcaggaaacacctgtggcctatctcacccgtgtcttccctggccagggaagggcagccgcttaaaggggccaagggaagctgcagcgtggctcctaaacatggacgcacagtagctgtcctctcaggagctctgcacccccacttaaaaccaggatcacaagagatttgtgtaaaggaagcctcttgtgcatttcttcatctgaatcgttgacttaaaagtttcttccacgtaagtcctttcactttagctttgtatatatttagcattgatttcacaacaaacaactgattccaggcacttggtgttttcccagaccttgagctttttccagggttgcacgttatttccatatgtggtacttcgcggcagccccagtatggggaagccatttgctgccttatgaattgtctggtcatccactcgagaggaaagggctgtgtccagggctggggaaggccccaagagtggttcttcaggcctactgttggcaacagcccaggaatgtgtgaagtctgtaacaacaagcgtagtaataataattccagctctgctagttgaacgctcactttgtgccagctacttttctaagagttccacgatatgaacaatttaatcctcaaagccaacatctgagaaagatactactattcttatcccaatttgacagatgagaaaactaaggctcagagaggcttgtctgctggtgatttgtgtgtctgggaagtgcacgttgtctggcaggcttgcctggcagaagcggttcatgacaggtcttctccattttggaatcaagatctgtgtcagcatggatggcaaggggaagagtaaggaaagattggtctaggagatgcccaggagcttttggcctcacagtgcagggtggggagagcagacctggccaccaaggaccagtttctgggcttgggcagaagccaatcatctggctactttacaagcagatggagcttcttcctctcagtaaccttcctgccctggtgtcaattccattttcattttcatcttttatggtactggttgtgtggagaaaactgaaggctcatttggtataagtaagtaactcatttttatgagtttgcttgacatagatactagactgtatgtgtgtatatatacatataaacatgcatatgtacataaaaaatatatatatatcatatatacatgatgatacacacacacacacacacacacacacacacacatatatatatatacgtatacaagcatgctttacaaggccaattgactggtctacaattggctgacacttggtggcctagaagccagggtatgtgagtctcgcttttctagaaagctgacaaactctccagttccaaggatccttgctcagtcaacggctggaagtcatttttacttcgctgttttttgtttgtttgtttgtttgtttttttagacaaagtctcattctgtcacccaggctagagtgcagtggcactatcatggctcactgcaatctccacctcctgggctcaagcgatcctcccacctcagccacccgagtaactgggactacaggtgcacaccaccatgcctggctaatttttgtatttttttagagacaaggttttgccatgttgcccgggttggtctcaaacccctgagcacaagtgatcctcctgcctcggcctctacaaagtgctggaattacaggtgtgagccactgcactcgatccattcttacttactttctttactttatttccaagcaaatgtttggagggaaaccaagagacttggatgcggccagccgaggcctttgggtttacaatcacaaatgtttttggtttgcccatgaaggcccaggctgcactctctgatgtcacaggaatcacctctcaaaccatgcaccaggtcttgaattcccttagggtgtgatctttagaggtccatctaggtatacccacccaagccattctttgactgctgacaggccttccttcataacaaggtgttccacagtccatttatatatggatgtcatctctgcccaccctgctgccaatttggttttctcccactcctggggtgtaaggcaagatgaaacatatcacatcccgttctaaactttattcttgtggccaggggtcagcaaactttttctgtaaagggccagatggcaaatatcttaggttttacaggccaagaagcaaatttggcatattatgtagctacttatatagtaaaataaaaatttccacaattatgtaattgatgaaactcaaaatgtaataataataatcgaaggcagtttttttgtagtataggtttaataatgagaagaatggaatcatttttggaggtgctaacattctgcttggttggaatttaaagttagtgttctgtatcagcaaatccattgccaatgttcatctaaaaatgttttcacttctgggccggatttcgttcaaaggctgcagtttgctgacctctgctcttggttacaccttttgaggcccttgctctccgagcataaaatggaatccatttatcagactaaatcgggaagattaaattttccagcctcacgaatgctcagccattgactcactcgttcatacaatgaacactcattgagcttatactacatgccaggtgctggaggaggcatggggcgcccaggagaaagatgctcgctttgcggccacagcccagtgggagggagacccatacctaccggtgctgtctcagaaacttgtggaacaaagatgaagcaatgttcatgttattcgcctacatctgtgaattacacaaggaagacgagtttgagaaatccgaagttcagtacaaatttatggtaacttttttaaaaaagaatacactgaagttttcttagtgaatggaataatgttccctttttctcccctgtacacacaaatacacaaaaactaacaaaaatacgtcgtgtgtgtctgatttgggttgtatttaaatcatttcataaatgactttttcccataacttcagtttcaaagttttaaagcacagtcaattaatgatttggcaacagctaagaaatcacaagttcccttcttttcatgtaaacttctgtaaaacacacgctacgttctgctgatggtaaatagagccatttcaggaagttagccagtttctcttctcggccacctcctgcatagagggtaccattctgcgctgctgcaagttacggaatgaaaaattagaacaacagaaacatggtaagccacttctatttctttagcaaagctttccaacagaatatggggtttctgacccagaaatctgggttggtggcaaatggtgtgagcctagaaagtaataaatgggcaaataaggataaaaattaaagatcgaaacaactgtaaatgcaggtaaagcggcttgctatgatctttaatttgtgcacacgttagtataaaggaattagagagtaaattttgaaaatcaaatgcagtgatgatcttactaatttggacaggaaaataagaaaatttcaagttagaaattgaactggaaatattacttactggccctaccagagacaatatcctcttccagaacaacagggttggaagagaaggtgagggaaatattcttcctttgctatttctgtagaaaaggacaaactctcttccttcacatacataggtcaattgctagatcctagtgaagcctgagcttaacctactgttggaggcttaaagttcgacattaattgctacttttcttggtcagagttttaaataattaggttggtacaaaaaactgtgattacttttccaccaacctaataacatgctacaatttctgtaattattattttacactgtcaagacatagcaggtggtccgtttttgttattgtcaagaactgtcagactaaaaatgaactttacacttctttttaaatgatacattttctagaaaattcaatgaggtttaagagcaattgaaaagtctgatttcaagagagtctcatccaaaatgtactatatatttttccccaaagtccttggagttaattttgacaacaatttaaagtacacttaagtcttttgaagttaatgggtctgccacccaggttggagtgcagtggcgtgatctcagctcactgcaacctccgcctcccgggttcaagcgattctcctgcctcaacctcccaagtagctgggactacaggtgtgtgccaccacgcctggctaatttttgtatttttagtagagacggggtttctccatgttggccaggctggtctcgaactcctgacctcaggtgatccgcctgtctcagcctcccaaagtgctgggattacaggcatgagccaccgcgcccggcctgaagttaatttttatacccacctaatgttcattatggatcttgaaggtaaattaattctgcactaaaattttacaatgctttacaaaatgactgtaggtggcccatatggaattcggtcaactgggccaatgacacatatgggattgcagttgaaattatccaattcctacttgatatttgtaagctgctgtgatagccagtataattgtactgtaagaatgtggtaaatagccggggcccggtggctcacgcctataatcccagcactttgggaagccgacgtgggcggatcacttgaggtcagtaggtagagaccagcccggtcaacacggcaaaacctcgtctctactaaaaatacaaaaattagccaggtgtggtggtacgcacctgtagtcccagctactcaggaggctgaggcaggagaatcgcttgagcccatgaggtggatgttgcagtgagcaaagatcgcaccattgtactccagcctgggcaacggagtaagactctgtttcaaaacaacaacaacaacaacaacaacagattggtaaatagagtaataataaaatcaaattaaacttgcaaaaaatggccactttgctcccactggtggccaatggaggtcaaggacctggctgacctcctgcctaaaggcagaggttgttagccttcgcaatggactcaaatcagagggggagctttcaaaactcctgctgcccagactgaaccccagatcaatgaaaccaaaatctctggatacagggcttggcatttgtagcttttagagttcctaagtatctctactgtgcagccaaagttaagaatcagtgccttagaacatcaacagttttttggtccttttgttaaaaagcacagtccgtttttttaggtggctagaaatgctccaggaagagctgaaatgtatttaccagccaccttggtttgattttagaaagcaaaatagaagttctaagtatgctttctctgaaaagctgagactgcagataagagtgagggcagttgatggagttcattctcctctttcaatcactgcttctcatcctttcattataataatctaagaatctcagagattatgaaagagaaagcagtcttatggaagaccccagactcacagaatattagggtgtgtttcacagggaaggatgtcattacccacagttagtctttgaaacgcagttggacattatttgtaagtgcatcatagtgtcgcctccaggttccattgaggggaacgtcattccaatgcaacatctctgagttcatctgggttattaaatggggttgagggatttgttatttttaaattagtagccccaatttaggactactcaagaccataggacaagcctgtccaaccctcggcctgcgggctgcatatggccgaggacagctttgaatgcagcccaagacaaattcataaactttctgaaaatattatgcatttgttttttagcccatcagctactgttagtgttagtgtattttatgtgtggcccaagacaattcttcttcttccagtgtggcccagagaagctgaaagattggacacccctgctataagacacagtaatataaatacataacctgtggttctggattggcattagcagatacaggctgtgttgattttgcagaaagttacaaagagctgctagttggtgtgtatgtctaaaatcagtagatttcctgtggttctaaggaatgacaaagaatctggaagttctctgtggtagcctgctcagtgcagaaagggaacgtggaaaatccgccaccagcatttgagtcttggaggttccacatagggctatcaggtctctgctgatcactgaaaccagatcatggccaactagccccttggcttcagccctcccaattcattaactactcaggtaaatctagggtcactttcaactctaccacctaccatctgagtgaccttgaaaacattcatctctctgagcctcaggtcccatgtctgtaaagcaggggcctcatggacttctttgggtttttttgtttttgtttttgtttctgaggattaaacaaatgctccctaccctatttcccagcatccagtaacacagtttttcatatttttgtgtatgttaagtcaggacccatctctttaatgataagtgcacttaatgtggtcatgttttcttttgtcttccaaagctgttagtgaatccattgaatttgggatgggtaaaataaagtatctattattaattgtaaatttcatctaaagtgacaaatcctacctgcataaccatttcttaatttcctttcatcatgtatcagtggtcaacattgttaactgcgaatgaatcagaatccatcaaaaattagaactatttccagtctggcaaaaattcagctctggttgaatccaaacattgtgctgaagcagctaagtaattcaactgaggagattaattacatgttataatcaataggttctcttgacacttcagtgttagggaacatcagcaagacccatcccaggagaccttgaaggaagcctttgaaagggagaatgaaggagtcatctttgcaaaatagctcctgcagcctgggaaaggagactaaaaaggtaaaaagctgttaattccaggaagacagctttacgcccctcccagaccacctgcactgcacactacgtggaatttattttagtctcacatggcagcgtccctacctttgtgcccacacatctggtctccgccctggctgcagccctccccttcaggcgaattctgggtgtgtcctatctgctcattgcaactcccagcgaatgagttttcagcgaaggcagactttctgacctgttcttcaaactgcactggtcttttaaaaacgtgtttggtggccatcagcatccaatttcagaagaaagatttgggtgaggactgagagaggctgttgttgttgtgctgtctgtttccttcagaatctgcagaagaaaattggcaggtcatgtactgtggacctaaccaaaggacaaatgatgtatggaaaatagaaaaactgttgtgaaattgcttcctcattagcaataactgtatttggcagggagaggagaagttgggcacatttttttttcttttttttttcatgattcatacgttttctttaaagaagtgggttttgcttttcactgggtgctctaagacaaccccagtgaaagatctggaccacgaagacccagtcatcctcataagggtgttcattgcagcaagctcaagggcatgccaggcaaaggccttttttctggcagcttgaacttgtctcagcagagggtttcacagaacaactgtcatttacctgttctctgctcttacttgattcgtttcccaggactgctgaaacaaagtaccacaaacttggtggatcaaaacagcagaaatatatcctctcacagttctggaaaccacaagtcagaaaccaatgtgttgttggcagggttggttccttcttaaggggctagagggaaaatctgtttcatgctcctctcccagcttctggtggtagctagcaattcttgatgctctctggcttgccgctgcatctctctagccttcacctctcctcatgtgggtggccttctttcctgtgtgtctatttccaaattccccttttcttataaggggaccagttattggatcagggcccaccttaattcagtagatcccattttaacttgatgacatcagcaaagtccaaataaggttgtattcacaggtaccaggggttagaacttcaagttatctattaggggacacaattcaacctaaaaactccccttttttgattctctattctgccacttctactcaatccaggttcttcacttcatcagctcccaatctaatacttatcttatttctagtaagcatctcttccttatcttaactggtccctggggcctggcccgagccccattataccatcagctgttgacatcaagggtggacttctctttcggcacagaaggcacagggctgtaggcttcagccttctctgctttgctctgccccatctactgttcatccacctgctttccattttgctaaactttgtagaaaattcttgtcagctgttgtctcctcctacactttctttgatcttagaggattctattcttttactatggctttaatcggagcacccgactgttaggttcaaccaacagaagttggttgtgctctctcactctttctttctctctctctctctttctctctatttgcatagtggtattttttttttcctctattttattggcagaattgccatttctctaagttattgtagagttgctgtttctctattttatttgcatatttctcttctgccaggctggattgtttctattgattggttctgctgtaatgagggtgacttctcattagtatccttctcacttcatctgggaccagatgccctttgatatccttttggagccacaacttttggtagtcagaggcatgggtgtggctcaaaggaagaacttggctcagaaggtgcagctcttgctgggcctttggtctctgctctgtcttctgagatcagtggctgctgggacctggggttcccccatgccgggcatggtcacacagcactcctatggacttgagcagagcaccctgcaaagtgagcattagcaatccattccaactctgtgcagtcctgcacggaatatagaaggtggagcaatgacagtctccccaacttctctgcaagcaacctgctcaccatttcttgcccttcccatttatgtacttttcaaaatcaggttatttggaatttgtcgactcatgtttcttacttcagtacttttttgggagggcagcattagaaacctcaaactcttaactaaaaaatgtctttgggaatgttctggccattttcatggcccacaatttgctttaagctgctttagactctcccagaggctattttcatcccgaaagaacagagcagagctcaaaagactccagttttggtctctagcagcccctagaggatttccccctcaattcctctctgccttgtatgaaatagaattggatttgaaatcggatgttgaggccttacctccaggctagtgaggccacacaagatggatcctctggacccgcccaagtgtccacctaaacatgagttaccaactaacaatgttttgtttagcatgcaaagggagtggtctggaatctggccttgccctgacatattctccttgggcctttttaaaaaaataatttgtgttaatctgtagttaaaaattataataaggacctgacaaacactacctcagtcagatgatcaaggtacacataaatagtgaaagtcatgttgatagcatgcacccttcatatgatatggctagaatggccctgcacttctgtgatcttcctcccctagactcatcagctcgatctaatcataacaaaagcatcagataagtccccgcccagggacattctacataaccatttcccttcccagttatatttttctccacaatactttccaccatctaacattctatctttcaaaatgggcaagtattttagcctggtttgttcattgttttatctgcaactcaaatacagttcctgaaataaaatatctgcctaataaatatttaatgaatgaatgaatatagcattgccttatccgtttaattgccacatggtatttcattgtgtgaacataatatcgtttatttacccagactactactcataggcatttagattatttccggtcttttgctattgctaacagcctttgcaatgaacatccttgtatacagacatttgcatatatgagggtgtgtctttaggatctacttctagaattgaaattgccaactccaagtatatgtttccaattgtgatagatattacacattaccctccatcttagaggtggtgttaatttagattcctgccagcaaaatttaagagtgtttgtttccccatatcctcaactgcctaacagaatcagtgaaaaatggtatgacagtgtaatttttgagtgaggttgagtatcttttcctatgctttaagagcaatttatgtttcctttttatgtgaactgtctgttaatatattttttcaatttttctattgggttatttgtcttttcattaatgcatatacctgttacatatttataccaagtatgtattaaatactaacatattgatgaaacagagcaaaaagcctagaaatagatccaaataacagaagagttagtatgtgatacaggaagcctataaaatcagtgagcaaaagaccatccaattaataacgttagggtaaatgggtctccatttagaaaaaaataatgtgggtctacacctcacattttatacctaaacaattccagtgggataagaaaatgaaatcataaaaaattactaggaaaaagatgagaaaattgttcataaaactgaagtgtggaagatcctttatgccttacactgccctgagtgatctcattcatacccatggcttcaattgtcatgaatcccaaattcattcctctgtcagaactctcttctgagcttcagacccacatactcagctgcctactggacacctctacttgaatatcacaaactcaactcaaaagcaaacctgtcaaatttaattactagtagccctaccccaaacaatcttcctgctcagtgaatgacacccatccctccaggtgcacagaccaggaacctagaagtcactctgattgcatccctctccctcacaacctctacctccctttattcatccattgctatgtctctcaaatgtacctcccaaatatctcttgaacgcgttcttttctatctctattgccaccaccctagttcaaactcccatcatctcatgactgaagttctgtgccctcttgccagtgaacactgtagaatcaatctaaacatggtgccaccctgcttaaaaaccttcaaaggctcacatcacttctcagatgaagagattggggagacgttggtaataggacacaaaatttcagttaggcaggaggaaaaagttctattgaagaactctattgtacaatatggtgactatagttaataacaacatattatacacttgaaaatcactaagagagtccattttaagtgttctcatgaccaaaaaatgataagtatatgaggtaatgcatatgtgaattagcttgactgaggcattctacatgtatacatatttcgaaacatcatgttgtacatcataaatgcatacactttttagttgtcaatttaattaatattttttaaacctactctggcctttttttccttttttgagacgggtggtctctgtcccccatgctagagtgcagtgcgcaatcatggctcactgcagcctccacctcccagtctcaggcgattctccagtctcagcctcccaagtagctgggaccacaagcatgagccaccatgccccgctatttgtttttgtattttttgtagagatgggatctcgccacatggcccagtctggtgtccaactcctgagctccagtgatccacctgcctcagcttcccaaactgctgggattacaggcgtgagccactgtgcctggtccactctggtctttactcaagtccctggctttctctcagtctcttaaacttatgtgcttagtaagatgaggactgaaaaatgtccacagaacatagtgacatggagatactgagaacctcaacgacatctccattagccacttcctctgtgccattccagtcctctgggccccactgtggcaagcagtcctaccatggcaaacatgaaagctgatgtgccttgtcttagacccacaccatatctctctgaattcctgtcccagggcttctctggaggtacagcctgggaaactcacgggaatagacacagggcctttgcacatgctgctcccttttcctgaaaaattcctttgacatcttggttgtgccttacacatgcctactcaaccttaggattgcagttcaggtttcactccttttttttttttctttttgagacggagtttcactcttgttgcccaggctggagtgcaatggtgtgatcctggctcaccacaacctctgcctcctgggttcaagtgattctcctgcctcaacctcctgagtagctgggattatagtcatgcaccaccacgcccagctaattttgtatttttagtagagacagtgtttctctatgttggccaggctggtctcgaactcccgacctcaggtgatcggcccgcctcggcctaggttccacttctttatggaaatcttccccagttgccttgactaggccaaagtcccctcttcttaggctcttacagtgtcatgcacttcttttttatcacagtgtaaaccttgtaatgttgtgtttaagtcatatctgttgtacccatgagactgggagccaattcatatattgtgagtgtaatcgaacagacttcccaggccacccactagctaatcaaggcagggatgagtccggaaagtgactttgaaatctagcaatgttggaacttggaaatcacacaggctgagatctgctcaggtgcctgaacaaatatagcattgcctgtggcgtctccctcaaagtgccttgcatgtctgagccccgttgccccttcctttggtgtgcctgtgtctcccggtacagatgtgaagcctggagacctgtggctgcctctgcaggagctccatgttttcaagccataaatcatcttagaattcatagcatctagatatattagttttctattactgcagaacaaatcgctcccaaatgtagaggcttcaaagaatgcccattgattggccttaatttctgtaagttagaatctgggcaggtttgcctgagttctccactccaagtctcataaagccaagctgggctgtcatctggaggctctgagtaaaaatttgtttccaggttcatccagattgtcaggtgatttcagttccttgcagttgttgttcgactcactaccccaccaccaccccgaaaacctcatttccttgctagctgcctgcagagagccactctcagcttccacaggctgcttgcattccttgttgtggggccgctacctcctcaagccagaaatagggcatccagttcttctcatgcatcctacccctctgacttttccttctgccgataaccagaaaaaacgttccgccttcaaacgctcgtatgattagactaagcccatccagataaattcccatatgccatatactataatgtcatcacagcagtaatacccgggacaaaattcatgggggtcatcttaaaattctgcctatcacaccaggtatagtagaggcttgttttagtgcaagttaaacattaagcagcaacatcacgatagtgctgcatttgaaaataactactagcaactgaacatgtctgggagttctgctccactttaatttccatctcaaaaggagctgggttttccttggctgttacaaatgggcaataatgattgagcttaagaataatcaatgtccacataaaaatcttttataacatagtgagagtgtgacatataaaggtgttagttcaccggccctaaattttaggagaatttttaaaaaggcacttatctggtttaatccataataaagacatgagttgggctttagtgaaaaatctaggctggtttctgtgttcagtgaaagaagatttgagagttctcttaattacaacccttgatcaaacctaccacattaatctgtttattgcattgtatggttaccaaaagtgatatattcagccctctatttattaagaaacagttacagaaagtgaggcactctcctgtgttactgagggtgcataaaaatataaagcaccatgtgtcttccctagagaagtttcaaaactagcaagcaaatagctattaatgctaatgtttgtgtgatagggaacatatgagtagtaattattccacaaacaattttttgagtgctgtttacatttgaggcacagttcaggcacgaggatttcaaaaggagattgtgtagcatgatggcttgttaaaaatatgattttggaatcagatttgctcaagtcccagtgctacagcataccatccttcaaaaaggtacttaagtctctgagtttgttttctcatctgcaaaatataaataataagaggacctactgcgtcatgttcttgtgagcattaatgtgggtgatgaaatgtttatgaagcacttagcacaatacctgacattttgtttgttattattatcaacataaagtgcccactttccagtcatgcaagaagaaaacataatatatgtcaccatagaagtatagaacaattgtgggaaataccagtaagagagatatagctgtataaataaggtaaagatgactgcctagaagatctaggatgataccatattagaagttgcatctgaactctccttggggactggccaaagtttcatcaagtgtcatgtcagtaggttggtgctataaatatatagcttgcaaagctatagacttactataaaccatagctgtggtccagcttagactcattatggtggtggagtatcttgattaatggcctctgcagaagcttcccaggtcttctcatcatcataatctcagatagcttcatcttcaacttccttttttttgttgtttttgagacagggtctcactctgtcatccaggatggagtgcagtggcacaatcatggctcactgcagcctcgacctcaggagctcaagccatcctcccacttcagcctcccgagtagttgggactacaggcatgcaccactacgcccggctaattttttcatttttttgtagagtcagggtctccctatgctgcccagtctggtctcaaactcctgggctcaaaccatctttccacctcggcctcccaaaatgttgggattacaggtgtgagccaccacacacagcccatcttcaacttcttttagcaccatgaagctgaacatagtaaaaaagtaaaatcattctggacctaatctgatgcaatttatttaattgttaagtgaatgcacacatcaaaattcatacaagtatggggcagcgctgctaatttatttacaaaacacctggcaaatactgctactctaatactgtgcttccacttttgattttccttaggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggatccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaactgacgttaagtcatgatttcgcgccataataaagatgcaaaggaatgacatttctgtattagttatctattgctatgtaacaaattatcccaaaacttagtggtttaaaacaacacatttgctggcccacagtttttgagggtcaggagtccaggcccagcataactgggtcctctgctcagggtgtctcagaggctgcaatgtaggtgttcaccagagacataggcatcactggggtcacactcatgtggttgttttctggattcaattcctcctgggctattggccaaaggctatactcatgtaagccatgcgagcctctcccacaaggcagcttgcttcatcagagctagcaaaaaagagaggttgctagcaagatgaagtcacaatcttttgtaatcgaatcaaaaaagtgatatctcatcactttggccatattctatttgttagaagtaaaccacaggtcccaccagctccatgggagtgaccacctcagtccagggaaaacagctgaagaccaagatggtgagctctgattgcttcagttggtcatcaactattttcccttgactgctgtcctgggatggcctgctatcttgatgatagattgtgaatatcaggaggcagggatcactgtggaccatcttagcagttgacctaacacatcttcttttcaatatctaagaacttttgccactgtgactaatggtcctaatattaagctgttgtttatatttatcatatatctatggctacatggttatattatgctgtggttgcgttcggttttatttacagttgcttttacaaatatttgctgtaacatttgacttctaaggtttagatgccatttaagaactgagatggatagcttttaaagcatcttttacttcttaccattttttaaaagtatgcagctaaattcgaagcttttggtctatattgttaattgccattgctgtaaatcttaaaatgaatgaataaaaatgtttcattttacaagaggagtgtatgataaatatatcatagagaaattggtctttaatataaaagaaattgccatatacactgaattttttcagaactctttttaaaaaactatttggtagaaatcaaaggggaagcagttttcatgacacttttactttaagatacttattaatagataaattctatcttgattccctactcagaagacataaagtcagaatgcctggctgttggtagcctttgtgcaattcccccaaatgaaacaactttggcaaccctttccacttctactgtccccttggttcctctgcatcagtccatagcatcctctatccagtatgaatcttgagatatctaatgaaatttacctgagaataactagaaattatccaagcataagaaaaggaagttgcttcagaatgaaaagaagataaacctccaatataccatctttcctttttagttaaatcttacagcatgagttaccttttaatatgtgcttctaagaaactgaccaaaataatgtgtcatagtgttatttaatacgcacaaagtggaaagcagtgcaagtttgccaaggacaatttaattttgtcacattgcatgctgttttgtgaccatgaagagtttatacaaagatgtttatgcttgtgcttgttgaggtatagggacaaatatctaaaagcaagatcagatgggtgtggtatctcacacctataatccttggattaaaatctacctcaattgtaggactaccagttgaaccacatgcttcccactgccctcagcaaagggcaccttagttagaggaaaggtagagcctttctatggaggaggaatttgtgaggtttgagttttatcagctacctgggagtcagaccctgatagattctccttcacactccctggaccttttcctgccaagtggaggctctcactcagaggaaatctccattcttttgatgcaggtcattcatactcagatattctgcactgttcaagcaataaaaattgaatgagcacctattatgtacaccagttggcactgtgtcaaaatgtacttgtgcagagaccttggatcattggtgacaggtcttcttctcctctgcatttttctcaagaccaggcctcagtgtagcatgtttccatggagtgaaagaggggaaggaagagtgggctttggaaagtggcagctgtgtcatagcagtcagcctctgtgtatgtgaaggactttccagagcccccccactaaagcctccatgctcctcctgggactgccacagttcttgaaactatccatacagtcttcatgagttatttttaatttttttttcttcttttctctttcctccttttccccttttccccactccctagttagatctttaaaaatgcaattgtaacctttatcttcccttcaccagacactccctacagggcaagcttatgtatacgcttacctaaaagctccagagccagaaatctctcccactcggggactgcctcaagagacagcagtcaatttacaacctaaagcatgcccacaacaaaactctctcccacctggaggatatcttgaggcaatggtcactttacaacctagttctgcctgcaatggcaccagctcaaccacctggtacataagacacaaaagcaagttgcatagacctcaccttctcactcccttccctgcatgccattaatgccaactccccctttaaaagcccctgctttctgccccaaaagcaaagtgatacccttaaagtcaggagcctatacttcttccccctaagctaatttttggaataaaagtcattttattgagaacctccataaactgttggtgggaatataaattagtaaaccatgatggagaacagtttggagtttcctcaaagaactaaaaatcgaattaccatatgacccagcaatcccactgctgggtatacacccaaaagaaaggaagtaattatattgaagagatatctgcactcccatgtttgctgcagcactgtttacaatagctgagatttgcagcaacctaagtgtctatcaacagatgaatggataaagaaaatgtggtacacatacaaaatggagtactagtcagccataaaaagaatgagatcctgtcatttgcaacaacatggatggaactggaggtcattatgttaagtgaaataagccagacacagaaagacaaatatcaaatgttctcacttatttgtgggatctaaaaattaaaacaattcaactcatggacatagagagtagaaggatggttaccagaggctgggaggggaagtggaagctaggggaggtggggatggttaatgggtacaaaaaaatagaaagaatgaattagatctactatttgatagcacaacagagtgactatagtcaataataatttaactgtactttttaaaaataacaaaaatcgtgtaattggactgtttataactcaaagaataaatgcttgaggggatggatatcccattctccatgatgtgattacccattgcatgtatcaaaacatctcgtgtaccccctaaatatatacacctactatgtatccacaaaaactaaaaataaaattttgtttaaaaagtcactttctttataccacatctcacccttgttaattggactctgcgaggggtgaacaactggacctgtgattcagttaaaattagatcctcaggcaccttctgttgagaaagaataggtctcaaatgttgcaaatctcttttacctttctcaaggttctagcctctcctatcaccaatttaggtaagaatataaaatcatcaggcctgtgttacctcaactatcttcctctttgatccaaaacgtatacttagtggacaaaggctttttggacaactaatatgtgctaagtcttaaggtgggttcagaaatggccaggatccatcaactaatcaatggataaataaaatgtgatctatccatacaatggaatattattcagccataaaatgtaatgaagcactaatactatgatgcaacatggatgaactttgaaaacatcatgctaatagaccaataacaggctctgaaattgtggcaataatcaatagcttgctaaccaaaaagagtccaggaccagatggattcacagccgaattctaccagaggtacaaggaggaactggtaccattccttctgaaactattccaatcaatagaaaaagaggggatcctccctaactcattttatgaggccagagtcatcctgataccaaagccgggcagagacacaaccaagaaagagaattttagaccaatatccttgatgaacattgatgcaaaaatcctcaataaaatactggcaaaccgaatccagcagcacatcaaaaagcttatgcaccatgatcaagtgggcttcatccctgggatgcaaggctggttcaatatatgcaaatcaataaacgtaatccagcatataaacagaaccaaagacaaaaaccacatgattatctcaatagatgcagaaaaggcctttgacaaaattcaacagcctttcatgctaaaaactctcaataaattaggtattgatgggatgtatctcaaaataataagagctatctatgaccaacccacagccagtatcatactgaatgggcaaaaactggaagcattccctttgaaaactggcacaagacagggatgccctctctcaccactcctattcaacatagtgttggaagttctggccaaggcaattaggcaggagaaggaaataaagggtattcaattaggaaaagaggaagtcaaattgtc

SEQ ID NO: 7 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 8 - DNA, 801 bp, Homo sapiens
acaggtgtgagccactgcactcgatccattcttacttactttctttactttatttccaagcaaatgtttggagggaaaccaagagacttggatgcggccagccgaggcctttgggtttacaatcacaaatgtttttggtttgcccatgaaggcccaggctgcactctctgatgtcacaggaatcacctctcaaaccatgcaccaggtcttgaattcccttagggtgtgatctttagaggtccatctaggtatacccacccaagccattctttgactgctgacaggccttccttcataacaaggtgttccacagtccatttatatatggatgtcatctctgcccaccctgctgccaatttggttttctcccactcctggggtgtaaggcaagatgaaacatrtcacatcccgttctaaactttattcttgtggccaggggtcagcaaactttttctgtaaagggccagatggcaaatatcttaggttttacaggccaagaagcaaatttggcatattatgtagctacttatatagtaaaataaaaatttccacaattatgtaattgatgaaactcaaaatgtaataataataatcgaaggcagtttttttgtagtataggtttaataatgagaagaatggaatcatttttggaggtgctaacattctgcttggttggaatttaaagttagtgttctgtatcagcaaatccattgccaatgttcatctaaaaatgttttcacttctgggccggatttcgttcaaaggctgcagtttgctgacctctgctcttggttacacctt

SEQ ID NO: 9 - DNA, 801 bp, Homo sapiens
aaaaatttccacaattatgtaattgatgaaactcaaaatgtaataataataatcgaaggcagtttttttgtagtataggtttaataatgagaagaatggaatcatttttggaggtgctaacattctgcttggttggaatttaaagttagtgttctgtatcagcaaatccattgccaatgttcatctaaaaatgttttcacttctgggccggatttcgttcaaaggctgcagtttgctgacctctgctcttggttacaccttttgaggcccttgctctccgagcataaaatggaatccatttatcagactaaatcgggaagattaaattttccagcctcacgaatgctcagccattgactcactcgttcatacaatgaacactcattgagcttatactacaygccaggtgctggaggaggcatggggcgcccaggagaaagatgctcgctttgcggccacagcccagtgggagggagacccatacctaccggtgctgtctcagaaacttgtggaacaaagatgaagcaatgttcatgttattcgcctacatctgtgaattacacaaggaagacgagtttgagaaatccgaagttcagtacaaatttatggtaacttttttaaaaaagaatacactgaagttttcttagtgaatggaataatgttccctttttctcccctgtacacacaaatacacaaaaactaacaaaaatacgtcgtgtgtgtctgatttgggttgtatttaaatcatttcataaatgactttttcccataacttcagtttcaaagttttaaagcacagtc

SEQ ID NO: 10 - DNA, 801 bp, Homo sapiens
acccatacctaccggtgctgtctcagaaacttgtggaacaaagatgaagcaatgttcatgttattcgcctacatctgtgaattacacaaggaagacgagtttgagaaatccgaagttcagtacaaatttatggtaacttttttaaaaaagaatacactgaagttttcttagtgaatggaataatgttccctttttctcccctgtacacacaaatacacaaaaactaacaaaaatacgtcgtgtgtgtctgatttgggttgtatttaaatcatttcataaatgactttttcccataacttcagtttcaaagttttaaagcacagtcaattaatgatttggcaacagctaagaaatcacaagttcccttcttttcatgtaaacttctgtaaaacacacgctasgttctgctgatggtaaatagagccatttcaggaagttagccagtttctcttctcggccacctcctgcatagagggtaccattctgcgctgctgcaagttacggaatgaaaaattagaacaacagaaacatggtaagccacttctatttctttagcaaagctttccaacagaatatggggtttctgacccagaaatctgggttggtggcaaatggtgtgagcctagaaagtaataaatgggcaaataaggataaaaattaaagatcgaaacaactgtaaatgcaggtaaagcggcttgctatgatctttaatttgtgcacacgttagtataaaggaattagagagtaaattttgaaaatcaaatgcagtgatgatcttactaatttggacaggaaaataag

SEQ ID NO: 11 - DNA, 801 bp, Homo sapiens
aatgttcatgttattcgcctacatctgtgaattacacaaggaagacgagtttgagaaatccgaagttcagtacaaatttatggtaacttttttaaaaaagaatacactgaagttttcttagtgaatggaataatgttccctttttctcccctgtacacacaaatacacaaaaactaacaaaaatacgtcgtgtgtgtctgatttgggttgtatttaaatcatttcataaatgactttttcccataacttcagtttcaaagttttaaagcacagtcaattaatgatttggcaacagctaagaaatcacaagttcccttcttttcatgtaaacttctgtaaaacacacgctacgttctgctgatggtaaatagagccatttcaggaagttagccagtttctcytctcggccacctcctgcatagagggtaccattctgcgctgctgcaagttacggaatgaaaaattagaacaacagaaacatggtaagccacttctatttctttagcaaagctttccaacagaatatggggtttctgacccagaaatctgggttggtggcaaatggtgtgagcctagaaagtaataaatgggcaaataaggataaaaattaaagatcgaaacaactgtaaatgcaggtaaagcggcttgctatgatctttaatttgtgcacacgttagtataaaggaattagagagtaaattttgaaaatcaaatgcagtgatgatcttactaatttggacaggaaaataagaaaatttcaagttagaaattgaactggaaatattacttactggccctacc

SEQ ID NO: 12 - DNA, 801 bp, Homo sapiens
atggtaacttttttaaaaaagaatacactgaagttttcttagtgaatggaataatgttccctttttctcccctgtacacacaaatacacaaaaactaacaaaaatacgtcgtgtgtgtctgatttgggttgtatttaaatcatttcataaatgactttttcccataacttcagtttcaaagttttaaagcacagtcaattaatgatttggcaacagctaagaaatcacaagttcccttcttttcatgtaaacttctgtaaaacacacgctacgttctgctgatggtaaatagagccatttcaggaagttagccagtttctcttctcggccacctcctgcatagagggtaccattctgcgctgctgcaagttacggaatgaaaaattagaacaacagaaacrtggtaagccacttctatttctttagcaaagctttccaacagaatatggggtttctgacccagaaatctgggttggtggcaaatggtgtgagcctagaaagtaataaatgggcaaataaggataaaaattaaagatcgaaacaactgtaaatgcaggtaaagcggcttgctatgatctttaatttgtgcacacgttagtataaaggaattagagagtaaattttgaaaatcaaatgcagtgatgatcttactaatttggacaggaaaataagaaaatttcaagttagaaattgaactggaaatattacttactggccctaccagagacaatatcctcttccagaacaacagggttggaagagaaggtgagggaaatattcttcctttgctatttctgtaga

SEQ ID NO: 13 - DNA, 201 bp, Homo sapiens
ctgaagttaatttttatacccacctaatgttcattatggatcttgaaggtaaattaattctgcactaaaattttacaatgctttacaaaatgactgtaggyggcccatatggaattcggtcaactgggccaatgacacatatgggattgcagttgaaattatccaattcctacttgatatttgtaagctgctgtgatagcc

SEQ ID NO: 14 - DNA, 801 bp, Homo sapiens
caacagttttttggtccttttgttaaaaagcacagtccgtttttttaggtggctagaaatgctccaggaagagctgaaatgtatttaccagccaccttggtttgattttagaaagcaaaatagaagttctaagtatgctttctctgaaaagctgagactgcagataagagtgagggcagttgatggagttcattctcctctttcaatcactgcttctcatcctttcattataataatctaagaatctcagagattatgaaagagaaagcagtcttatggaagaccccagactcacagaatattagggtgtgtttcacagggaaggatgtcattacccacagttagtctttgaaacgcagttggacattatttgtaagtgcatcatagtgtcgcctccaggwtccattgaggggaacgtcattccaatgcaacatctctgagttcatctgggttattaaatggggttgagggatttgttatttttaaattagtagccccaatttaggactactcaagaccataggacaagcctgtccaaccctcggcctgcgggctgcatatggccgaggacagctttgaatgcagcccaagacaaattcataaactttctgaaaatattatgcatttgttttttagcccatcagctactgttagtgttagtgtattttatgtgtggcccaagacaattcttcttcttccagtgtggcccagagaagctgaaagattggacacccctgctataagacacagtaatataaatacataacctgtggttctggattggcattagcagatacaggc

SEQ ID NO: 15 - DNA, 801 bp, Homo sapiens
ctttgaatgcagcccaagacaaattcataaactttctgaaaatattatgcatttgttttttagcccatcagctactgttagtgttagtgtattttatgtgtggcccaagacaattcttcttcttccagtgtggcccagagaagctgaaagattggacacccctgctataagacacagtaatataaatacataacctgtggttctggattggcattagcagatacaggctgtgttgattttgcagaaagttacaaagagctgctagttggtgtgtatgtctaaaatcagtagatttcctgtggttctaaggaatgacaaagaatctggaagttctctgtggtagcctgctcagtgcagaaagggaacgtggaaaatccgccaccagcatttgagtcttggargttccacatagggctatcaggtctctgctgatcactgaaaccagatcatggccaactagccccttggcttcagccctcccaattcattaactactcaggtaaatctagggtcactttcaactctaccacctaccatctgagtgaccttgaaaacattcatctctctgagcctcaggtcccatgtctgtaaagcaggggcctcatggacttctttgggtttttttgtttttgtttttgtttctgaggattaaacaaatgctccctaccctatttcccagcatccagtaacacagtttttcatatttttgtgtatgttaagtcaggacccatctctttaatgataagtgcacttaatgtggtcatgttttcttttgtcttccaaagctgttagtgaatccat

SEQ ID NO: 16 - DNA, 801 bp, Homo sapiens
cataacctgtggttctggattggcattagcagatacaggctgtgttgattttgcagaaagttacaaagagctgctagttggtgtgtatgtctaaaatcagtagatttcctgtggttctaaggaatgacaaagaatctggaagttctctgtggtagcctgctcagtgcagaaagggaacgtggaaaatccgccaccagcatttgagtcttggaggttccacatagggctatcaggtctctgctgatcactgaaaccagatcatggccaactagccccttggcttcagccctcccaattcattaactactcaggtaaatctagggtcactttcaactctaccacctaccatctgagtgaccttgaaaacattcatctctctgagcctcaggtcccatgtctgyaaagcaggggcctcatggacttctttgggtttttttgtttttgtttttgtttctgaggattaaacaaatgctccctaccctatttcccagcatccagtaacacagtttttcatatttttgtgtatgttaagtcaggacccatctctttaatgataagtgcacttaatgtggtcatgttttcttttgtcttccaaagctgttagtgaatccattgaatttgggatgggtaaaataaagtatctattattaattgtaaatttcatctaaagtgacaaatcctacctgcataaccatttcttaatttcctttcatcatgtatcagtggtcaacattgttaactgcgaatgaatcagaatccatcaaaaattagaactatttccagtctggcaaaaattcagct

SEQ ID NO: 17 - DNA, 21803 bp, Homo sapiens
cggaggcagcgagaaagcgcagccaggcggctgctcggcgttctctcaggtgactgctcggagttctcccaggtacgtcgtgcgctccccactcgtgtggtctctctgcaccccttcctggggtcgggtcgggcaggggcggaggggagccgcagcccggtcacgggctctggggaacccgggctcccgttcggctgcacctgggcccctagctcctgtccgggcggggatagcgggaagcgcaccaggcccccgggacgccggtgcttctttgcacggcccagcgcgggaatgtggacaccagcgaagccggctcaggtcggccctcgggagaagcaggggctacacggaaagggagcccgagggtcctgcagagcccgaggtcgggaccccgcgcgtgcagtgccaatgggctgcggctgggctgagcgcccttccaaccttgcaggccccgggggggttgccgcggagagcttccgctggggctccgggcctctggccgccacctccctcgggaggccgaggggggcggtggccccggcccggccctcccgcttccctcacggcctggccctcccgcttccctgattcccggtagggtcgctctccccgcctcctggcccctcttggctcggatgacaaagattgggtaaaggctttcaacgagctagcgtctggcagggggtgtgtcctcgctcactggttactgctaggacggagcttggattttggtcaccgtggctcagaggtgggaaaggttccgtgctctgggtctgtccaggaccctggctccctaacccaagtctgacctctgctccctggcaggcttcgccccacctctgcgttccttcattcttaggatgtgtgagttaggtctctgattagaaaacaacttacagcgtttaagcaaagtcgagtccttcaagtaagaatgctttgaagtgccctttacaacttcttcaacttctataaatatgaacttgtgtttagaacttaaatgcaagccaggaaagtctttttcctgtttgaaagagagagaaagagagagaaagaggtgattggaagtggtgggtgggggcggagaaagagaaggagagtaatctgggtgtagacaactcaaagtttgctgagatgactttaatcttttgactctgttattaagatttttaccaaagagcaactgtagctatggtggcctgacttgaagggacagaccaaacaaacaacaaaacacaaaacaaaacaaacaaacaacaaaagcaagagcagaaactattggaaaatgatttattcaccaatattttaaaggttcttcctgttctaagcaaggaacatctttaagttctttataatgtacccattttacatttacaaaaactgccagaagttgacagcatgttttgggggtttctaagctatcagttaattttttggggtacagttttattgataaactaacagagtagaaaaaatctaatggaatacccccaaatttaaaagagggcaagaaaagagagacaatagaacataaaacaaatgggacaagaataaagtacatagttgtcacagtcccttgggtgctgctgtaacaaaatacctgagactgggtaatttacaaagaacagaaatttatccattcatggttctggagtctgggaagtccaagattgaagggctgcatctggtgagggtcatctggctacattataacatgatggaaagcatcacatggtgagagagagcaagagaggacagaacttacttttataacaaactcattctcacaataaactgctcccttggtgataacattaatccatttatgagggtacagccttcataacctaatcacctcttaaaaggttccacctctcagctcgcagtgagccgagatcatgccactgcactccagcctgggtgacagagcgagactctgtctcaaaacaaacaaacaaacaaaaattccacctctcaacactgttgcattgaagatgaaatttccaatacatgagctttggggcacacattcaaaccatagcaatagttcaattactttaaagcccaaaatgtcagtaattatattaaatgcaatttaatacatcagtaactaaatgagatagactaaattttatgataatcagactaatattatcagactagacttcaaaacaacccaacaatcatatgctgtttacaagagacacatttatggatactaaaaggtataaagtgaacagatgcaaccatttatcaaaagaaagctgttgtggcaatattactattagacaaaatagaattaggaaaaaaatcataactgtagataaacgtttcactaagttaaaggtttcactgagaagaaataaaaattttgaatttataagtacttataacaacattaagaaactagaaaatagaaaaagaatagagccaaattgactgaactagaaaaatagaaaattcatagaaaaatccacaatcatagtgggagattttatcatacatctctcagtaactgatagaaaaaccagacaaaaatcaataaggataaagaaaatgcgaacacaatgtagttcacataaggaacctatactacttccaacagcagctgaaatcacagagcatacttaaaatctgaccatatactggctcatagagtaaattgcaatacattttaaagaatttaaatcatacagaatatgtcttctgaccataatgcaattaaagtagaaaagaacagaaaaacttatagaaaattctcatatatttggaaattagtaaatttctaaataacccatgggtcaaaaaataatcagaatgaaaatcagaaaatatttttaatgacttatgaaaaaaattacatataaaaacttgtcagatgcacctaaactgttgcttaaaggaaacttacaaccttaagtgcatatattagaaatgaagagtgacgaaaaatgaatgagcaagcaaataccttaaaaactggaaaaggaatagtaaaataaatccagagaaatcagaacaggggaaataataatataagaatagaaattaatgtgctagaaaataggggatctatcaagtcagaagttgattttttgaaaatgctaataaaaatgatgaacctctggcaagacaataaaagctggtacaaataactcatctaaggaataaaattgaggacctctcttttgttaaatgacataagcaggaggccattatcctgagactgtctctacactttgagttcttacataataaactgcaacctaatttagtatactacataaacaaactgaaacctaacttaggagctttttttttgtaattaatagctaaatttccaccaaatcataaatagccaagctttagccaatcacaggcagccaactgatcagactatgcccaaacaaggtaaatgcctattacaccatacccaaataaggcagactcctagctgaagccaatcaggtgattactatgctcttgtgtttggcctataaaagctcactgctcacactgttggatgctctctgaaacttttgtgaatctgagtgctgcctgattcatgaatcattctttgctaaaataaactctgtcaaatttaatttgtctgaagtttctcttttaacagtgtttggtgttgcaagcaggatccaaaggagacctatagtgactcccaggagctcttagtgaccaagtgaaggtacctgtggggctcattgtgcccattgctctttcactgctttcaactggtagttgtgggtaagttcctgctcagacttgagcactatacatttgcattttcagctctctgactttatttgagcaattctttgatggattggctccagaattggattggattcagtgaagaaactgaaactggactggtgctctaggaggcctcaagtagataaggttactagaagacagggaatcctgggcttatctggatttaaggagtctacaactccatctgcaactccagccgatttcatttataagaattatggaccaagaacctgtgcttttctagagaaatgtgtgagccttactaaaggtaacttagaattacaatggactgctgagaccagctcggtcgtggagaccctaacccagcagcgctagaggaatcaaagacacacacacagaaatgtagagtgtggagtgggaaatcaggggactcacagccttcagagctgagagcccctaacagagatttacccacatatttactgacagcaagccagtgataagcattatcgctatagattatagattaactaaaagtattccttacaggaaacaaagggatgggccgaaacaaagggataggctctggctagatgtctgcagcaggaacacgtccttaaggcacagattgctcatgctattgttgtggcttaggaacacgttaagcggttttctgccctgggtgggccaggtgttccttgccctcattccagtaaacccacaaccttcagcgtgggcgtcatggctgtcacaaacatgtcacagtgctgcagagattttgtttatggccaaatctggggctagtttatggccaggtttgggagcctgttcccacatgtcccccttttttgttttgcaaagcaataaaagcaaaggcagctttgtcacagtgagctacttcttgcaggagtcgggattcgcatatgcatactatacaaagacaaacaacacagattaaaagcacaaccatcattgaaatcacagagcttccaagtgtctttatctattttaatgggttaatcactgctaatctgtctgcagctccttcaagcactccagttcctggcattaaggtctggtgtgcctggaatgctttaaatatttgttcttttaatattgcaatatggaaagacaagtttgtagagtgtcttctaggtgcttttttattctttcctgaatttggtcttatttaagagccattaatagtttccacaaatccttatgtttagctcctacagcgggccatatcatttgagggtgaggtgccactataccatcatgtttccagataataggaactcttgccatatttcttgctatttctaccatctgaccattttgtttagaccagctaaacatagtgtggccatggcacgcagactgagaggtgcaatttaagctaaacatccccttaggggaccaatcaataatgattccataggaatcactgcgcagcacctctgcctattctgcaatgcaatcttcccaaacaagtatgttcattatttctggccaggtccaattctgtttacaaataggtttttgagggcaatatgcctcaattatagaaacagatttattatggtaaatactgagatcagaaagcatgtgtaactgtgtcatagaatgattacatccaggcattattgccagccaagattgataaatatgcccaataagtataattgttctctgtgttagcccttgttgaaggaatactaatggcaatggtgatcactgctatcatagctatcattaaattactcattgtgactggttgtcccactttcctcaggttttcttccaccctctatggcagtttcttgatctgtccccaggtagatggctgtgtttgatggttgttgctcgtgatagtttgggtcctcctcagcatcagtctcgacatggctgcaaccagagggtcctcgggatcctcccagaatctcttccttggcatctggctcatgataaggtttcaggtgtcttgatggtatccaaatcagctattgattctggcctggagaaacacaagcataacctctaccccaagttattattttacctatttcccaactttttgttgtcagatctctgcaccaagccagttgttctgcttctgtttttgcagctggtttctgtagatgctcttcagctgctgatagcatctggcctttaggcaggctcaaaaaatttaaagtcattaatgctagattcagttgtatatggggtgtcctgtaatccctgtttcccctttttgcttttgcaactgctgtttcagggagagattcattctttctacaatggtttgtccttgagaattatatgggatgccagtaatgtgtttaatattccatatagagaaaaatgtagctagagcttggctagtatagcctggggcattatctgttttaacagaagttggaattcctatcaccacaaaacactgcaaaaggtgatgcttaacacatgcagtctcctgattggcatgtagcccagacaaagtgagaaaaggtgtccacacatacatgtacataagctagtctcccaaacgagggaacgtgtgacatccatttgctaaagataattaggttccaatcctcgaggattaactcctcctgttaaagatgaggaatgcaccatttggcaagttgggcatcactggataatagctttagcttctttccaggtactgttatatctgcatttgagaccagaggcattaacatgggttaaattgtgaaagtgtctagcattagatattgcagtagcaactaggcgatcagccatttgattcccttcagttaaaggtcctggaagaggtgtatgagccctaatgtgagtgatgtaaaaagggtgcattctactcctaaatgctgtttgcaattgggtaaataaagtcatcagttgttcatctgtatgaagtcataactgagcattttcaattaattgtatggaatgaaccacataggaagaatcagaaatcacattaataggcatatcaaaagcagtcaatacctcaattacagctaaagctccactttttgagctgaagtatagggcgtgtgaaaaactttaccttttgatccagaataagaggctttaccattacaagacccatctgtaaaaacattctcagcaccttcagttggtttaaatttagttattttggggagaatccaattagttaatttcaaaaattgaaataatttcattttaggaaaatgattatcaagaatacccacaaagtcagctaaatgggtttgccaagtaagactatttatagaagcttgctgtatttgtgcctttgtgagagggacaataatttttccaggatcatatccatgtaatttaacaatccgaattctcccatttcttatcatcgtagccatttgatccaaataaggagttagagtctgtgaattagtatgtggaagaaaaagccactgtacaaagtcttgctcttatacaataacaccagtaagtgaatgctgagttggaaaaattagcaaatctagggtcttttttggatctattctatttatttgagatttatggatttgctttttgattagctgcagctctgcctcagcttcttttgttaattgctgagggctaatgagactaggatctcctctaaggatagaaaatagattactcagggcataggtgggaatgcctagagcaggtcatatccgattaatgtcccctagtaatttttgaaagtcatttaatgttttcaattgatccctacgtatagttactttctgtggcacaatggtagtgtcatttactaaggtccccaagtaggagtaaggagtagtagtctgaattttgtcaggagctatataattaaactggcatgagaaatcgagttttgcaagtgatcataacattggagtaatatttcttgagtgggggcagcacaaagtatattatccatataatgaataatgtaacactgtgaaaattttttacgagtaggttcaattgcttgcccttcataagtctggcaaatttttgaactgtttcacatgccttgtagcaacactttccaatacaaatgctttagtatgctgcaggttttttactgcaggaattgtaaatgcaaacagttcacagccttgctcagctaaggggatagtaaagaaacagtcttttaaatttatgactattaaaggccaattttttggaatcatagcagaagaaggcagtcccggctgcaatgtccccataggttgtataactgaattaatggctcttaagtcagttaacattctgcatttacctgattttttcttaattacaaaaactggagaattccaaggggaaaatgttgagctatgtgtcctttttctaattgttcagcaactaagtcctctaaagcctccagtttctctttactcagcagccattgtgctatccaaattggcttatctgttaaccattttaaaggtatataggttctggaggcttaacaatggccaccatcaaaaatgatatcctaaaccttggtgggaactttgtctttccgcttgaagcatttccttcaaagcttgcaaattttttcctagtcccataccagggacataccccatttcatgcatcatatgttgactttgagggctgtataattattctggaattagaacttgtgatccccattgttgtaataaatctcttccctataaatttataggtacagaagttataattttttgtatagtcccaggttgtccattgggcccttcacaatgcaaaatataactactttgatatactttaagggctttaccaactccaactatgttaaattgagcaggttgaattgaccatgcagatggccagtgctgtacagaaatgattgaaatgtccgctcctgtatctaccaaacttttaaatttctttccctgaatagttatttcaccagtaggatgtttatcagtaatttgattcacccaataagctgctttgccttgtttatttgtgcttccaaatcttcctgttcatttaatttcacttttccccatttccacatatggcataatcaggagctgtgctatatgctctcctggctctgctttccagggaacagaagtagatataacaatttgaatttcccctttgtaatctgaatcaatgactcctgtttgtacttgcacgccttttaaatttaaactagacctacctagaagtaatcctaccatccccgctggcatcatctgactggagacagcaaccttctttaacagtcccattacaaaaggagaacctgttccatatggattaatagcttgtttaaattctttgagtaatttaaaaggaaaaggctcaaatgtagctataatatttccctgttgatctacggggtatattctaacagggaactgctaagcctctatatcaccctctcttctaacttgctgaattcctggctgaatagaactgagagtggtcgctcgaggtgctgctcagtcactggggcgactactttttgcccagtgtcctctggaaaagaaagatctggaggatcaggctactcttttgcttcgaaataatgagggggtgcagaagggtagggacaaacctctccctcctttgccactttagctttagctggcaaacaaacctgctctgtcacctcttctgttacttcattatactttccttcctcctcatctttagtgtgaaaaggttccaaggtggaatgagctagagcccacacttgcccattgttaccctgatgcttccgagctccccttcttactcaccatggggattgcttaagagtactcgggtatcctccagctttgttccacgttttccaaccattgctccagcgagccttcatcctgggtttgagtccccacgttgggcgccacttgccgagaccagctcggtcgtggagaccctaacccagcagtgctagaggaattaaagacacacacacagaaacatagagtgtggagtgggaaatcaggggattcacagccttcagagctgagagccctgaacagagatttacccacgtatttattgacagcaagccggtgataagcattatttctgtagattatagattaactaaaagtattccttacaggaaacaaagggatgggccaaaacaaagcgatgggctctggctagatatctgcagcaggaacacgtccttaaggcacagatcgctcatgctattgtttgtggcttaggaatgccttaagtggttttccatcttgggtggaccaggtgttccttgccctcattccagtaaacccacaaccttcagtgtgggcgtcatggccatcacaaacatgtcacagtgctgcagagattttgtttatggcaagatctggggccagtttatggccagatttgagggcctgttcccaacagtggacaaagaagaaaagctgcttaaggcaaacaggcacatattttttaaagctacaatagctagacaaactaagagaatcctgtgtatgttttacttttgtatttttatttttaatatttgtggatacatagtagatgtatatatttatggggcacttgagatattttgatacaggcatacaatggataataataacatcacggtaaaaggggtatccattacctcaagcatttatcctttctttgtgttataaacaatccaattaaaccttttatcataaaatgtacaataatgttgactgtggtcaccctgttttgctatcaaagaataaatcttattcattttatctaagtatatttgtgtacccattaaccatcccagtccccctgctcctccaactacacttcccagcctctgttaaccatcattctactctcttatctccatgagttcaattgttttaatttgtagctcccacaaataagcgagaacatgggatgtttgtctttctgtgccttatttcacttaacataatatcctccagttccatccatgttgttatatgacagaatttcattctttcttatggctgaatagtaccccattgtatatatataccacattttctttatccattcatgtgtaggtggacacttggattgcttctagatcttggctactgtgaatagtgcagcactaaacatgggcgtgcagatatctccttgatatattgatttcctttattttgggtatatacctagcagtgggattgctggattgtgtggtagctctactgttagtcttttgaggaacctccaaattgttctccatagtggttgtactaatttacattcccaaccaacaacagtgtacagggttctgttttctccacatccttgcagcatttttattgcctgtcttttggataaaagccattttaactggggtgaaatgatatctcattgtagttttgattagcatttctctgatgatcattgatgttgagcacctttttatatatctgtttgccattcatgtgccttcttttgagaaatgtctatttagatcttttgcccatttaaaaaattggattattagatttttttcctattgagttatttgatctccttatatattctggttattaatcccctttcaaatggatagtttgtgaatattttctcccattctgtgtattgcctctgcactttgttgattatttaatttgctatgcagaaactttttaacttgatgtgatcccttttgctcatttttgctttggttgcctatgcttgtggggtatgacttaagaaatctttgcccagaccaatgtcatggagagttttcttaatgttttcttttaatagtttcatagtttgatgtcttagatttaagtctttaatctattttgtttttatttttgtatatggcaagatataggggtctagtttcttccgcatatggatatctacttttcccagtaccatttgttgaagagactgtcctttctgcaatgtatgttcttgacacttttattgaaaatgaattcactgtagatgtgtggatttgtttctgggttctctattctgttctgttgttctgtgtgtctgtttttatgccagtaccattctgttttagttactatagctctgtagtataattttaagtcagataatgtgattcctccagttttgttctttttgctcagaatagctctggctattctgggtcttttgtggtttcatgtaaattttaggattatattttattaacatatttctgtgaagaatgtcattggtattttgataggaactgcattaaatctatagattgctttgggtagtaaggatattttaacaatattgatttttccaatccatgaacatggaatatctttccattttttggtgtcctctttaatttcttgcatcagtgtttatagtgtttctttcatcagtgtttatagttttcattgtagagatcttttactcctttgattaagtttattcctgggtattttatttgtagctgttgtagacgagattactatcttgatttctttttcagattgttcactgttggcatatacaaatgctactgatttttgtatgttgattttgtatactacaatttcactgaatttgcttattacagagagactaaagatagttaggtccattaccaaatgtgttttctgtcacattgaaaaattataccatgagaaggcacatgcttctagtaattacagttcatagatatgtcaatgtacagaatgctggtgtgacagttcacaattgtttgctttatagttttcactggaaattaaattactaagggtaaagtaatcccagcactgtgggaagctgaggcaggatgattgcttaatgccaggagtttgagaccagctggggcaatgtagcatgagcccatctacaaaaaaaaaaaggaattctaattaatatgtggtactagaattaccagaaataataagggaaacaactcggaaaagaaggtaagatgtgttttgggtaagaacagctataaagtatgaggggatgttttgttttgttaaggaaaaaataaaataatatttgtcctaaagtagaatgcctggttgttaagacaatgagaaagagaaagagtatagaagaaaaactgaatggatataagaaagttgtagaaggttgtgggataggaatcctggaaagggaattttatatgtgatcaaactaagatttgaagaaaattctaagtttttttttaaaaaaattgagcatgaatatcaaaaacatactgacacagaactagaaatttgtcctctatgttaaaacaacaaggttttcttggagtattggtctgctcttaataagaaattgaaagacttttctttacctgctaagtaattggcctaggaaatgaagattctgtgttttattgagataatttattgtgcttcatgttgtctttattagatctttgactgcctcagagacctgagttcaccttattaaaagagaaagagtttctacaattatgtggctttctttatttgcctttgaagtcttttaccaatcactctagttaaatgaataaatattgtttcatagggacctgtgatcctattttaatcagatattttaaaccttttgatatttttgacttctcaaaatcaaattctaaattaaaaccttttgacgttgaacaaactttggaagcttctagatgggcttctggaatatctcaaaagaaattgttttctctcctgataaaaaagagagatattaaactaattgggcttatttgatatgttatattgcatgggaagaattgttaaattataagtgatgctgaactttctttaagtcatattgtatgggtatgttattaatatgtgcttcagaaattatatgaaattcttagaaaccagatagtcgtggtataatactatcagccaaaattctaattatcgtgaaatattatgtgttgcagaagtaatacaatttccttatcaattgcattattgtgaactctcatcatatctttaaccaagtccattttaagctttgtcatctacaaagagtcacttgttttactctggtactttccggaacactgtgaaaaacaattatataaagggtgtggacctataagacaaacaaccaaacaaaaatcgtaaagtgttcttttaaaagattcgtggaaaggactctgataagtacaggtttctgataactttaagatcatactactggactgggtaagaatttccagaactctagcaaagaagtggattggttcataaaactgccaacccaacatgaagcagaataagaattaattgaataccagggaaatgctttggcagattttcatgctaagtcagccagtactgaaattgttaagatatgcaatttgaaagaacactataatattcatctgaatcaaattatctatgataacctatttaaatgaacagcatcatgcacctgaattggagaaacaaaactgatatttaaaatgatataaatttagtgttaaatgtggcctcatggatcagacagctatctggtcctttctgagtccatatagctgccattattaaaagttctgcactctatgactcatcacagaatagataacgtgaaattatgaaaaaatattggtgttatgactgttctaaaattgctaaaatggcttataatcaatgtttggtttgtcaaatccacaatttttataagataataaaaacctcagttggtacatttccaccatccactggactatttgaacatttacagatggattgcattcagttgcctcattgggaatctgcaggaaacttaagtaaatgctggatatgtcatgttgaaccaaatactaatatgcttttaaagatcttctaattcaccctatagcagatttcactgatattccaagtgtgactacctgttcaaattgcacatctggtctttctatagggttaggctttttgagtcacatattcagatttcatgtttaaagctaacagtagacaaaacatataggagaggctttacagttaaattattccagaaactgaataataaaaccaaaaggagagataattagacagtttgtagacgaattcataaccctaattctctaattgttggtagcctccattgatgataattaatagtggagccttagataaatactactattgcttccccgttgatgacccctgtagcacacagtgtcccacagggaactgtctcttgtgcccctctagcatatatttttatctatgaaggatttaactatcaatcataggcacaggcaactccatgtcccagtcagtaggaaataagaagtcagtgtggattagggattctaacagtaccactgtcactccataaccaactgaaaatttgacattgttctatacctcttaatttgcactatggtataaaaaggaactttctagcaggtacgaatccctctaaatgggcatcttttgttcaaatgctccttccctggcttggaataaatgtaaatgaggttatgattagaaatccccttcaaacattagctactacagctcactctactgcaaaggctatatagttgcccagtgaagttctctaaattctcttgctaaagtttctttaaataacaggattgccttgaactatctgttggtttgtagtggggcagggggtgtgataggggacactgtgtataatagctaatacatcctactgcacttgcataaaggaatctgttataaagaaaatgaaataaaggtataaaagttggcaagaagaaaaccatcattatgtcttgaggatgattggggatacagaaaatgagaagaatccacaaactattagaattactaagttaatttagcaaggttgctcaatgcatggtcagtatacaaaatttaattgaatttctacatatatgtatatgccatattgtgttagattgttttgcattgctataagggaatacctgaagctgggtaatttataaagaaaagaggcttatttggctcatggttctgcaggctgtacaagcatggcactagcatctgtttgaccttaggaagcctttactcatggtggaagaagggggagcaggcatgtcatatagtgagagagggagtgagagggagggagcaagagtgaagggggaggtcccagactctttttaaataacctgaaatcctgtgaacagagtgaaaactaacttattaccatgaagagcacaccaagccattcatgaggggtataccttcatgacccaaacgctttccaccaggtcctaccttcaacactggggatcacatttcaacatgagatttggagaggacaaacatccaaatcatatcacatatataaatatgtaaaactattttaaatgtttttaaagtatcgaatagaaataaatctcacaaaatatgcttaagaactttaagcaaaaaccataaaatactattgtgataagttaaagaagacctaaataaatggagagatctgtcatgctcatggattggaagattcaatactgtaatgatatgaatagttcccaaattgttctatagattcaaaacattgccagttaaaatctcagtggactttgtttttttgaaactgacaagctgattctgccatttataataatattcaaaggggcaagaataaccaagacacatttgaagaagaacaaggaggaagaagaacgtatttgaagaagaataaggaactctattaagtgtatatctcctatatattgagatacacatttgaaatacatctatcacctagatattgagatatattatacatttaatatagtgtgttgctggtatagggatagacaaatgaacattggatcagaatagcactgaaacaagatctacccctatatgggcatgtccctattaacaaaaaaatattgttgattatttcctcaagatgatctgtaaaaaacaagttaattgaacatccatatgggaaaaaagtgaatccttctgctacctcaaatatcaaccccaggtagattatagatctaaaagtgaaaggcaaagcaataaagattttagaaaataacaaagagaatatctttatgaaaatacaggaaatattttaaaaataggctagaaaactcactattaaaatatataaaatataagaacatctgttcatcaaaagtactatagtttgaatagcagtgtctcctccaaaattcatattgaaacttaatccccattgtaacagtactaatcctaaaactaagtattaagaggtgtgacctgtgggaagtgactaagtcatgaggggtctttcctcaggtatgggattagcatccttataaaaggccttgagatggaatgtagtgcccccttgccctcttgcgttctcccatgtgaggacacagcaacaaggcaccatcttggaaggagagagaagccctcaccagacaacagtgcacatgccttgatcttagacttcccagcctccagagctataataaatacatttctgttctttataaattacttggtctctggtattttgttacagaagcactaatggactaagacaaaaagattccattatgaaagtgaaaaggcaaccgcagagtggcagaagcttttggagatgatgaatatgtttacttcctggattgcggtgatgataccatgagtgtatacatatgtgtgtatacataatgtatacaaattgtgttcattgattatgtacagtttctttgtataccaattataccttaataaagctaaggaaaaaaaaaagaaaatccagaataaatatgcatggtatgacccatttatttaaaaaaaaaaagagggaatgcgaagcagtggttttggtgcgaacacaaaaattctggcttcaacagcatttaaattcccaaaccaagcatgtttgtgttttaattctttgtggccataaattgtacagctcaggcctttatagtctctcagattctgtgaatgtggggaattagttttactcataaaaagttttgttcttggggataaattttttaaaaaaatttttgtatagttagcacactgagaaaatacagacaaaggcatacagatgcaagaaatgaggtagctgaatagaccaaagaatgatatagggcccagaaggtaggcaaagagaaagttgttgggtttatggttacaagtaaactagcagttgtggtgcagatagttttatttcccccacattaatctgaacagcccatccagacttaaacactgctttttgcatttacttctaggcaggaagacagggttctgatggtgtgagtctccttcaactcagcaaaccaccttggtctgcctcgagtttccaacacccctcctgcctgctagtgataggtgtgaggcaggttgatgaacatggaacttttttcttttggtcccaaagcatgctactcctggagtttcattcagtgaatgagaactatagtttggttctgtgagatctctatgaatcaaggcggccactgaagcggagaaaagaaatgcttaaatgttaagaaagtttgaagtgcagaaaaaggtgattgtaaatccatatggttaagcttagcccatttcttaaaaggcttgattgctcattcctccattcattgatttactcactcttccaatccatgttattgagtcttgctctgtaattccggatggttgtgcttttaagtactgcatagtggttgtatgtctgtgttagcattgctgaatgtatcagggaattcatttttttatccccattcattcgttccattcatctgtttctctctctctctctctttgtgtgtgtgtgtgtgtgtgtgtgtgtgtgtgtgtgtgtgtgtgttatgcctagaaaacatttctcaagaattagaattacgatatgctgtcaaacacaatgacttatttgaacctcttttatttgtaggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgaggacttgcttactaaaactacaaaacttcaaattttgtctggggtgctgttttataaacatatgccagatttaaaaattggtttttggtttttcttttttctatgagataaccatgatcataagtctattactgatatctgaatatagtcccttggtatccaagggaattggttgcaggatcctcgtggatatcaaaattcatagatgatcaagtcccttataagagtggcatagtatttgcatataacctgtgtacattctcctgtatactttaaatcatctctagattacttatgatacccaatacaatgtaaatactatgtaaatagttgtactgtctttttatttatattattattgttattttttattttcaaaatttttaaaacatacttttgatccacagttggttgacttcatggatgcagaacccatggatatagagggccaactgtaatctgtagcaactggcttagttcattaggaaacagcacaaatgaacttaagattctcaatgactgtgtcattctttcttcctgctaagagactcctctgtggccacaaaaggcattctctgtcctacctagctgtcacttctctgtgcagctgatctcaagagcaacaaggcaaagtatttggggcactccccaaaacttgttgctattcctagaaaaaagtgctgtgtatttcctattaaactttacaggatgagaaa

SEQ ID NO: 18 - DNA, 201 bp, Homo sapiens
ctgttcctgctgatcctgctcacgggggtcctgtgccaccgtttccatggcctgtggtatatgaaaatgatgtgggcctggctccaggccaaaaggaagcmcaggaaagctcccagcaggaacatctgctatgatgcatttgtttcttacagtgagcgggatgcctactgggtggagaaccttatggtccaggagctggag

SEQ ID NO: 19 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

(19) United States
(12) Patent Application Publication
(10) Pub. No.: US 2010/0021917 A1
(43) Pub. Date: Jan. 28, 2010

(54) METHODS OF USING GENES AND GENETIC VARIANTS TO PREDICT OR DIAGNOSE INFLAMMATORY BOWEL DISEASE

(75) Inventors: Jerome I. Rotter, Los Angeles, CA (US); Kent D. Taylor, Ventura, CA (US); Stephan R. Targan, Santa Monica, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/527,376

(22) PCT Filed: Feb. 14, 2008
(86) PCT No.: PCT/US08/54033

§ 371 Date: Sep. 1, 2009

Related U.S. Application Data
(60) Provisional application No. 60/889,806, filed on Feb. 14, 2007.

(51) Int. Cl. C12Q 1/68

(52) U.S. Cl. 435/6

(57) ABSTRACT

This invention provides methods of diagnosing or predicting susceptibility to inflammatory bowel disease by determining the presence or absence of genetic variants. In one embodiment, a the invention is practiced by determining the presence or absence of NOD2 variants in an individual where the presence of NOD2 variants are indicative of susceptibility to Crohn's Disease in the individual. In another embodiment, the invention further determines the presence or absence of TLR8 variants where the presence of TLR8 variants are inflammatory bowel disease in female individuals. In another embodiment, the invention further determines the presence or absence of TR2 variant P631H where the presence of TLR2 variant P631H is indicative of susceptibility to Crohn's Disease.

### FIELD OF THE INVENTION

The invention relates generally to the fields of inflammation and autoimmunity and autoimmune disease and, more specifically, to genetic methods for diagnosing inflammatory bowel disease, Crohn's disease, and other autoimmune diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments provide methods of diagnosing susceptibility to Crohn's Disease in an individual, comprising determining the presence or absence of at least one risk variant at the NOD2 locus selected from the group consisting of R702W, G908R and 1007fs, and determining the presence or absence of at least one risk serological marker, where the presence of at least one risk variant and at least one risk serological marker is diagnostic of susceptibility to Crohn's Disease.

In other embodiments, the presence of three of the risk variants at the NOD2 locus present a greater susceptibility than the presence of two, one or none of the risk variants at the NOD2 locus, and the presence of two of the risk variants at the NOD2 locus presents a greater susceptibility than the presence of one or none of the risk variants at the NOD2 locus but less than the presence of three risk variants at the NOD2 locus, and the presence of one of the risk variants at the NOD2 locus presents a greater susceptibility than the presence of none of the risk variants at the NOD2 locus but less than the presence of three or two of the risk variants at the NOD2 locus.

In other embodiments, the risk serological markers are selected from the group consisting of ASCA, I2, OmpC and Cbir. In another embodiment, the presence of four of the risk serological markers presents a greater susceptibility than the presence of three or two or one or none of the risk serological markers, and the presence of three of the risk serological markers presents a greater susceptibility than the presence of two or one or none of the risk serological markers but less than the presence of four risk serological markers, and the presence of two of the risk serological markers presents a greater susceptibility than the presence of one or none of the risk serological markers but less than the presence of four or three risk serological markers, and the presence of one of the risk serological markers presents a greater susceptibility than the presence of none of the risk serological markers but less than the presence of four or three or two of the risk serological markers.

In another embodiment, the invention further comprises the step of determining the presence or absence of one or more risk haplotypes at the TLR8 locus, wherein the presence of one or more risk haplotypes at the TLR8 locus is diagnostic of susceptibility to Crohn's Disease.

In another embodiment, the invention comprises the step of determining the presence or absence of one or more risk haplotypes at the TLR2 locus, wherein the presence of one or more risk haplotypes at the TLR2 locus is diagnostic of susceptibility to Crohn's Disease.

Other various embodiments provide methods of diagnosing susceptibility to Crohn's Disease in an individual comprising determining the presence or absence of one or more risk haplotypes at the TLR8 locus in the individual, where the presence of one or more risk haplotypes is diagnostic of susceptibility to Crohn's Disease. In other embodiments, the individual is a female. In another embodiment, the method further comprises determining the presence of H3.

Other various embodiments provide methods of determining a low probability relative to a healthy individual of developing Crohn's Disease and/or ulcerative colitis in an individual, the method comprising determining the presence or absence of one or more protective haplotypes at the TLR8 locus in the individual, where the presence of one or more said protective haplotypes is diagnostic of a low probability relative to a healthy individual of developing Crohn's Disease and/or ulcerative colitis. In other embodiments, the individual is a female. In other embodiments, the method further comprises determining the presence of H2.

Further embodiments provide methods of diagnosing susceptibility to Crohn's Disease in an individual comprising determining the presence or absence of one or more risk variants at the TLR2 locus in the individual, where the presence of one or more risk variants is diagnostic of susceptibility to Crohn's Disease. In another embodiment, the individual is Jewish. In another embodiment, the invention further comprises determining the presence of P631H at the TLR2 locus.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts quartile analysis of the CD cohort for the 4 tested microbial antigens (ASCA, I2, OmpC, and CBir1). Reactivity to each antigen was divided into 4 quartiles and a value ascribed to a given individual based on their quartile of reactivity to each antigen (left panel). Quartile sums were calculated by the addition of the quartile value for each antigen (range, 4-16). The distribution of quartile sums is shown (right panel). Values for binding levels are in enzyme-linked immunosorbent assay units except for ASCA, which is presented in standardized format. Quartile sums were calculated similarly for unaffected relatives and healthy controls based on the distribution within each group (the quartile cut-off values and the distribution of quartile sums for the other two groups are not represented in this figure).

FIG. 2 depicts the frequency of carriage of any NOD2 variant increased with qualitative antibody reactivity, as represented by the antibody sum (number of positive antibodies, range 0-4). The dotted line represents the 31.8% frequency of carriage of at least one NOD2 variant, across the entire cohort.

FIG. 3 depicts the frequency of carriage of any NOD2 variant increased with semiquantitative antibody reactivity, as represented by the quartile sum (range, 4-16). The dotted line represents the 31.8% frequency of carriage of at least one NOD2 variant, across the entire cohort.

FIG. 4 depicts the cumulative semi-quantitative antibody reactivity, as represented by mean quartile sum, increased with increasing number of NOD2 variants by trend analysis (P=0.002).

FIG. 5 depicts the cohort of CD patients divided into mutually exclusive groups based on all possible permutations of antibody positivity: no positive antibodies, single antibody positivity (4 groups in set 1), double antibody positivity (6 groups in set 2), and triple antibody positivity (4 groups in set 3), and all antibodies positive. Within each of the three sets, where the groups had the same number of antibody positivity, there was no statistically significant difference in the frequency of NOD2 variants among sets 1, 2, and 3, respectively.

FIG. 6 depicts the cumulative semi-quantitative antibody reactivity in unaffected relatives of CD patients, as represented by mean quartile sum, was higher in individuals carrying any NOD2 variant than those carrying no variant (P=0.02). The quartile sum in unaffected relatives is based on quartiles of sero-reactivity within this cohort specifically and is not representative of the same magnitude of reactivity as an equivalent quartile sum value in a CD patient or a healthy control. No individuals carried two variants.

FIG. 7 depicts the cumulative semi-quantitative antibody reactivity in healthy controls, as represented by mean quartile sum, was numerically higher (though not achieving statistical significance) in individuals carrying any NOD2 variant than those carrying no variant (P=0.07). The quartile sum in healthy controls is based on quartiles of sero reactivity within this cohort specifically and is not representative of the same magnitude of reactivity as an equivalent quartile sum value in a CD patient or unaffected relative. No individuals carried two variants.

FIG. 8 depicts TLR8 haplotype associations with corresponding SNPs. As described herein, the data demonstrates that H3 (“211”) is a risk haplotype associated with Crohn's Disease in females, and H2 (“222”) is a protective haplotype against Crohn's Disease in females. “2” is the major allele, and “1” is the minor allele.

FIG. 9 depicts TLR8 haplotype associations with corresponding SNPs. It should be noted that Haplotype H3 spans two listings from HapMap data, and H1 has a minor component noted as ( ).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein refers to a set of single nucleotide polymorphisms (SNPs) on a gene or chromatid that are statistically associated.

“Protective” and “protection” as used herein refer to a decrease in susceptibility to IBD, including but not limited to CD and UC.

“Risk variant” as used herein refers to an allele whose presence is associated with an increase in susceptibility to IBD, including but not limited to CD and UC, relative to a healthy individual.

“Protective variant” as used herein refers to an allele whose presence is associated with a decrease in susceptibility to IBD, including but not limited to CD and UC, relative to an individual diagnosed with IBD.

“Risk haplotype” as used herein refers to a haplotype sequence whose presence is associated with an increase in susceptibility to IBD, including but not limited to CD and UC, relative to a healthy individual.

“Protective haplotype” as used herein refers to a haplotype sequence whose presence is associated with a decrease in susceptibility to IBD, including but not limited to CD and UC, relative to an individual diagnosed with IBD.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, the term “sero-reactivity” means positive expression of an antibody.

As used herein, R702W, G908R, and 1007fs variant alleles are also described as SNP 8, 12, and 13, respectively, as well as R675W, G881R, and 3020insC, respectively.

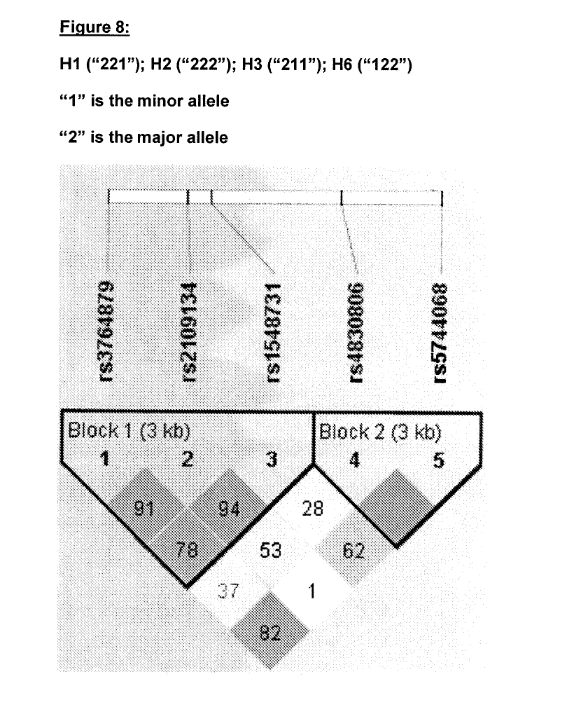

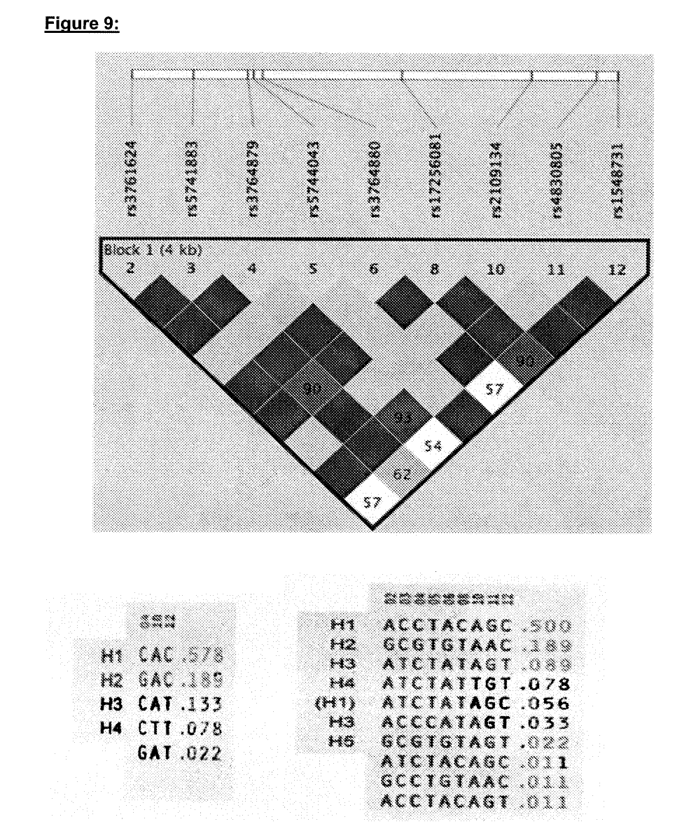

As used herein, the term of “TLR8 H3” is further described in FIGS. 8 and 9 herein.

As used herein, the term of “TLR8 H2” is further described in FIGS. 8 and 9 herein.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD and UC. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals, predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and/or ulcerative colitis. Other embodiments provide for methods of prognosing inflammatory bowel disease including but not limited to Crohn's Disease and/or ulcerative colitis. Other embodiments provide for methods of treating inflammatory bowel disease including but not limited to Crohn's Disease and/or ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

### NOD2

As disclosed herein, the inventors studied the serologic and genetic (NOD2) characteristics of a 732 patient cohort (Table 1). ASCA is detected in 50.4%, anti-12 in 58.1%, anti-OmpC in 37.2% and anti-Cbir1 in 56.4% (Table 1). Simple heterozygosity for a disease-predisposing NOD2 variant is detected in 194 patients (26.5%), compound heterozygosity for two NOD2 variants is detected in 23 patients (3.1%), and homozygosity for two NOD2 variants is detected in 16 patients (2.2%) (Table 1).

TABLE 1
Serologic and Genetic (NOD2) Characteristics of the Crohn's Disease
Patient Cohort

| | Cohort |
|---|---|
| Serologic and Genetic Characteristics | (n = 732) |
| Serological profile (%) | |
| ASCA positive (N = 369) | 50.4 |
| Anti-I2 positive (N = 425) | 58.1 |
| Anti-OmpC positive (N = 272) | 37.2 |
| Anti-CBir1 positive (N = 413) | 56.4 |
| NOD2 genotype for R702W, G908R, 1007fs (%) | |
| No mutations (N = 499) | 68.2 |
| Heterozygous (N = 194) | 26.5 |
| Compound heterozygous (N = 23) | 3.1 |
| Homozygous (N = 16) | 2.2 |

As disclosed herein, an example of a NOD2 genetic sequence is described as SEQ. ID. NO.: 1. An example of a NOD2 peptide sequence is described herein as SEQ. ID. NO.: 2. R702W, G908R, and 1007fs variant alleles are also described herein as SEQ. ID. NO.: 3, SEQ. ID. NO.: 4, and SEQ. ID. NO.: 5, respectively, wherein the position of the variant allele is marked within the sequence listing as a letter other than A, C, G or T.

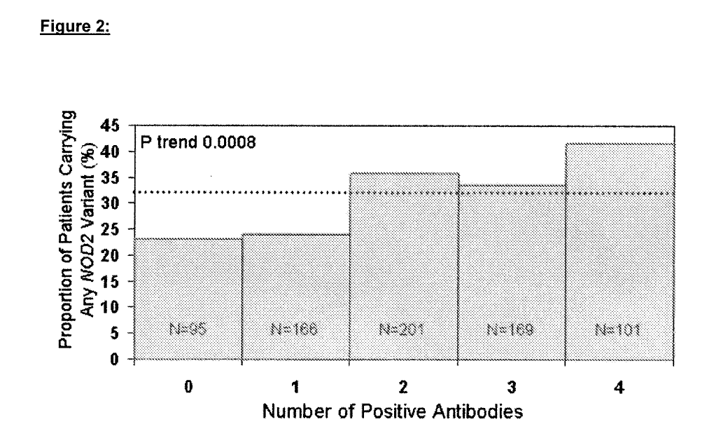

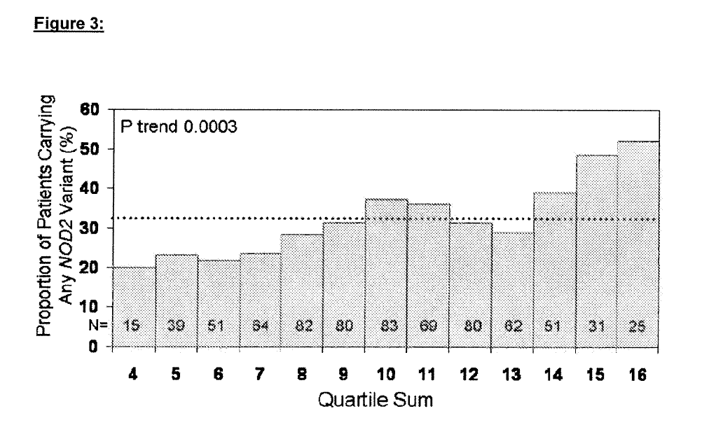

As further disclosed herein, a Crohn's Disease patient cohort was divided into five groups based on the number of antibodies (from zero to four) for which they are qualitatively positive and the proportion of patients with NOD2 variant in each group is determined. NOD2 variants are present with increasing frequency in patients with reactivity to an increasing number of microbial antigens, especially when there is reactivity to two or more antibodies (FIG. 2). NOD2 variants are present in those with 0,1,2,3 or 4 positive antibodies at a frequency of 23%, 24%, 36% and 42 % respectively (P for trend=0.0008) (FIG. 2). NOD2 variants are present at increasing frequency in patients with increasing cumulative semi-quantitative immune response as reflected by individual quartile sums (P for trend 0.0003) (FIG. 3). As the serologic response is increased, either qualitatively (by number of positive antibodies) or semi-quantitatively (by magnitude of the cumulative serological response), the likelihood of a patient carrying a NOD2 variant is increased (FIGS. 2 and 3).

As further disclosed herein, the inventors compared the serologic response of patients carrying a NOD2 variant to those carrying no variant. In patients carrying any NOD2 variant, the mean number of positive antibodies is higher than in those carrying no variant (2.24± versus 1.92±1.24, respectively; P=0.0008) (Table 2). Patients carrying any NOD2 variant have a higher mean quartile sum than those carrying no variant (10.60±3.03 versus 9.72±3.01, respectively; P=0.0003) (Table 2).

TABLE 2
Cumulative Qualitative and Semi-Quantitative Sero-reactivity to
Microbial Antigens According to NOD2 Variant Status in
Crohn's Disease Patients

| | No NOD2 Variant | Any NOD2 Variant | |
|---|---|---|---|
| | (n = 499) | (n = 233) | P-value |
| Mean number of | 1.92 +/− 1.24 | 2.24 +/− 1.21 | 0.0008 |
| antibody positivity | | | |
| Mean quartile sum* | 9.72 +/− 3.01 | 10.60 +/− 3.03 | 0.0003 |

*Mean +/− Standard Deviation

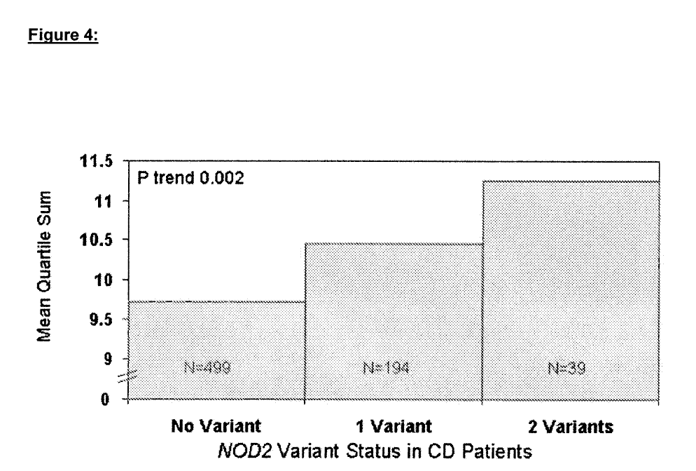

As disclosed herein, the inventors compared the serologic response of patients with two defective alleles versus having only one. The mean quartile sum increases in parallel with increasing number of NOD2 variants (P trend=0.002) (FIG. 4).

As further disclosed herein, the inventors examined the absolute level of response to each antibody individually rather than collectively. For each of the four antibodies, the magnitude of sero-reactivity is higher when a NOD2 variant is present (Table 3).

TABLE 3
Median Sero-reactivity to Individual Microbial Antigens According to
NOD2 Variant Status in Crohn's Disease Patients

| | Median | | |
|---|---|---|---|
| | seroreactivity in | Median seroreactivity in | |
| | EU/ml* (range) | EU/ml* (range) | |
| Antibody | No NOD2 Variant | Any NOD2 Variant | P-value |
| ASCA* | 0.032 (−1.40-2.31) | 0.620 (−1.26-2.57) | <0.0001 |
| Anti-I2 | 25.00 (0-248) | 27.56 (0-324) | 0.04 |
| Anti-OmpC | 16.32 (0-147) | 20.14 (0-203) | 0.03 |
| Anti-CBir1 | 28.36 (3.01-257) | 33.83 (0-280) | 0.01 |

*Sero-reactivity toward ASCA is expressed in standardized units with a mean of zero and a standard deviation of +/−one, thus a standardized unit may have a negative value

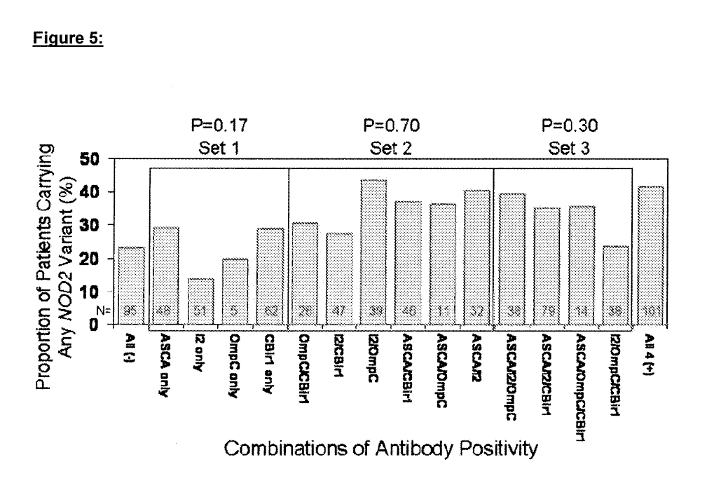

As further disclosed herein, the inventors divided Crohn's Disease patients into 16 mutually exclusive groups based on all possible permutations of antibody positivity: no positive antibodies, single antibody positivity (4 groups in set 1), double antibody positivity (6 groups in set 2), triple antibody positivity (4 groups in set 3), and all antibodies positive. The inventors tested whether there is a significant difference among groups within each set where the groups had the same number of antibody positivity. There is no statistically significant difference in the frequency of NOD2 variants among groups within each set, and no single antibody or combination of antibody positivity is wholly responsible for the association between sero-reactivity and variant status (FIG. 5). As disclosed herein, the inventors discovered that the relationship between NOD2 variants and serologic response to microbial antigens reflects a cumulative effect rather than being driven by any particular antibody or antibody combination.

As further disclosed herein, the inventors calculated 2.7% as the proportion of variability in sero-reactivity that was attributable to the presence of a NOD2 variant.

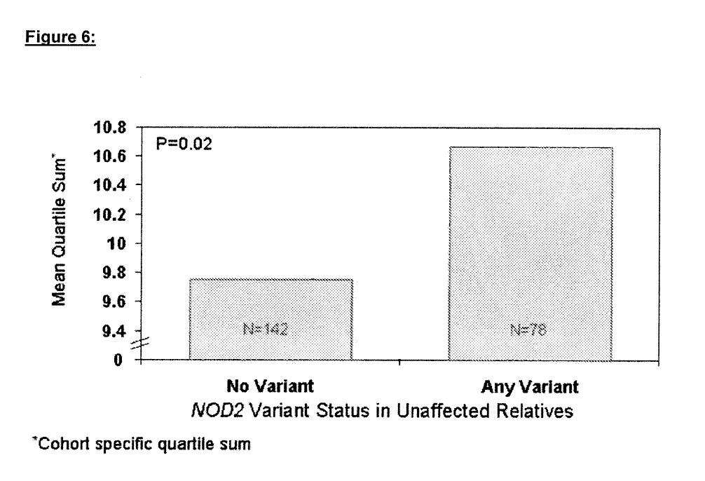

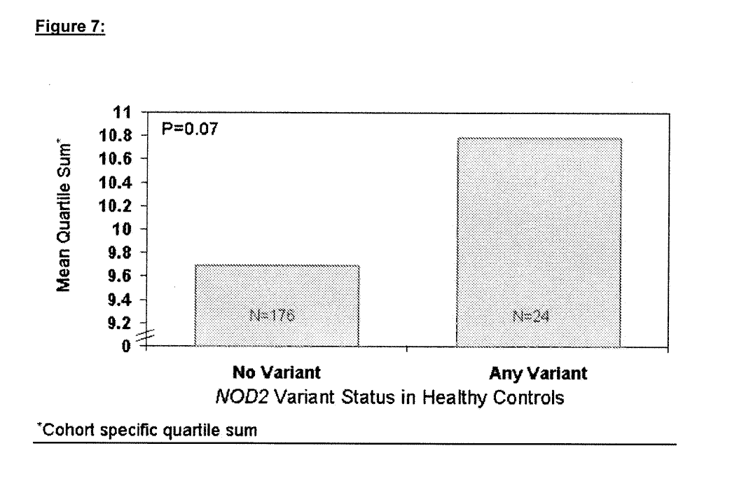

As further disclosed, a quartile sum was derived in Crohn's Disease patients, unaffected relatives, and healthy controls, based on the distribution of the magnitude of sero-reactivity within each cohort, with the same quartile sum in a Crohn's Disease patient or in a relative or healthy control not representative of the same absolute magnitude of response and not directly comparable. The magnitude of serologic response is significantly lower in unaffected relatives and healthy controls, compared to cases, and generally fell within the normal range. Sera was utilized from 220 unaffected relatives of Crohn's Disease patients (92% first degree). In the unaffected relatives the mean quartile sum in those individuals carrying any NOD2 variant is higher than those carrying no variant (10.67±2.73 vs. 9.75±2.52; P=0.02) (FIG. 6). Sera was utilized from 200 healthy controls. The mean quartile sum in healthy controls carrying any NOD2 variant is higher than healthy controls carrying no variant (n=176) (10.79±2.95 vs. 9.69±2.71; P=0.07) (FIG. 7).

NOD2 is a member of a family of intracellular cytosolic proteins important in mediating the host response to bacterial antigens and is found in epithelial cells of the small and large intestine as well as monocytes, macrophages, T and B cells, Paneth cells and dendritic cells (39-42). NOD2 senses MDP, a highly conserved component of bacterial peptidoglycan, which leads to the secretion of anti-bacterial substances such as alpha-defensins and the activation of nuclear factor kappa B (NF-kB) (43-44).

In one embodiment, the present invention provides methods of diagnosing and/or predicting susceptibility to Crohn's Disease in an individual by determining the presence or absence in the individual of R702W, G908, and/or 1000fs in the NOD2 gene. In another embodiment, the present invention provides methods of prognosis of Crohn's Disease in an individual by determining the presence or absence in the individual of R702W, G908, and/or 1000fs in the NOD2 gene. In another embodiment, the present invention provides methods of treatment of Crohn's Disease in an individual by determining the presence or absence in the individual of R702W, G908, and/or 1000fs in the NOD2 gene.

In another embodiment, sero-reactivity associated with NOD2 variants is diagnostic or predictive of susceptibility of Crohn's Disease. In another embodiment, the association of sero-reactivity of ASCA, I2, OmpC, or Cbir to variants R702W, G908R, or 1000fs, is diagnostic or predictive of susceptibility of Crohn's Disease. In another embodiment, the association of sero-reactivity of ASCA, I2, OmpC, or Cbir to variants R702W, G908R, or 1000fs provides methods of prognosis of Crohn's Disease. In another embodiment, the association of sero-reactivity of ASCA, I2, OmpC, or Cbir to variants R702W, G908R, or 1000fs provides methods of treatment of Crohn's Disease.

In another embodiment, the presence of R702W, G908R, or 1000fs NOD2 variant is diagnostic or predictive of an increased adaptive immune response.

### TLR8

As disclosed herein, the inventors examined a case-control cohort consisting of 763 Crohn's Disease patients, 351 ulcerative colitis patients, and 254 control patients. The patients were genotyped using Illumina technology. SNPs were chosen to tag common Caucasian haplotypes using information from the Innate Immunity PGA.

As further disclosed herein, both a “risk” and a “protective” TLR8 haplotype is associated with CD in females (risk haplotype (H3): 18% of CD subjects have H3 compared with 8.9% of control subjects; protective haplotype (H2): 59% of CD subjects have H2 compared to 72% of control subjects). No significant association with TLR8 and CD in males is observed. H2 is also associated with UC in females (59% of UC females have H2 compared with 72% of controls, p=0.024) as well as males (32% of UC males have H2 compared with 47% of controls, p=0.009).

TLR8 haplotypes as described herein utilize data from the published Innate Immunity PGA collaboration.

As disclosed herein, an example of a TLR8 genetic sequence is described as SEQ. ID. NO.: 6. An example of a TLR8 peptide sequence is described herein as SEQ. ID. NO.: 7.

H2 and H3 are further described herein by FIGS. 8 and 9, noting which A, C, G, and T variant corresponds to the listed reference number. These aforementioned listed reference numbers rs3761624, rs5741883, rs3764879, rs5744043, rs3764880, rs17256081, rs2109134, rs4830805, and rs1548731, are also described herein as SEQ. ID. NOS.: 8-16, respectively, wherein the position of the variant allele within the sequence listing is marked as a letter other than A, C, G or T.

In one embodiment, the present invention provides methods of diagnosing and/or predicting susceptibility for or protection against inflammatory bowel disease in an individual by determining the presence or absence in the individual of a haplotype in the TLR8 gene.

In one embodiment, the present invention provides a method of determining susceptibility and/or diagnosing Crohn's Disease in an individual by determining the presence or absence of a TLR8 risk haplotype. In another embodiment, the TLR8 risk haplotype includes H3. In another embodiment, the individual is a female.

In another embodiment, the present invention provides a method of determining protection against Crohn's Disease in an individual by determining the presence or absence of a TLR8 protective haplotype. In another embodiment, the TLR8 protective haplotype includes H2. In another embodiment, the individual is a female. In another embodiment, the presence of a H2 determines protection against ulcerative colitis.

In another embodiment, the presence of H3 and/or H2 may provide methods of prognosis of inflammatory bowel disease. In another embodiment, the presence of H3 and/or H2 may provide methods of treatment of inflammatory bowel disease.

### TLR2

As disclosed herein, the inventors tested sera from 731 CD patients (282 J, 449 NJ) for ASCA, anti-I2, anti-OmpC, and anti-CBir1 by ELISA while DNA was tested for five TLR2, two TLR4, and two TLR9 variants. The magnitude of responses to microbial antigens was examined according to variant status. Overall quartile sums (QS) (ranging from 4-16) of levels for all four antibodies were calculated as previously described (Mow et al Gastro 2004; 126:414).

As further disclosed herein, there is no association between any TLR4 or 9 variant and sero-reactivity to microbial antigens in Jewish or non-Jewish patients with CD. There is an association between the non-synonymous, non-conservative P631H variant of TLR2 and ASCA positivity in Jewish patients (OR 2.75, p for interaction=0.01). There is an association between the P631H variant of TLR2 and cumulative quantitative response to microbial antigens in Jewish patients with CD. QS is clustered into four groups by increasing cumulative quantitative immune response (group 1=4-6, group 2=7-9, group 3=10-13, and group 4=14-16). The frequency of carriage of the P631H variant of TLR2 increase in parallel with QS cluster in Jewish patients; 2.86%, 3.70%, 7.02%, and 13.46% in groups 1, 2,3, and 4, respectively (p for trend=0.03). No similar association is found in non-Jewish patients; 7.14%, 10.42%, 6.67%, and 5.45% in groups 1,2,3, and 4, respectively (p for trend=0.40).

As disclosed herein, an example of a TLR2 genetic sequence is described as SEQ. ID. NO.: 17. An example of a TLR2 peptide sequence is described herein as SEQ. ID. NO.: 19.

The P631H variant of TLR2 is also described herein as SEQ. ID. NO.: 18, wherein the position of the variant allele within the sequence listing is marked as M.

In one embodiment, the present invention provides methods of diagnosing and/or predicting susceptibility for or protection against Crohn's Disease in an individual by determining the presence or absence in the individual of a variant in the TLR2 gene.

In another embodiment, the P631H variant of the TLR2 gene is diagnostic or predictive of susceptibility to Crohn's Disease.

In another embodiment, sero-reactivity associated with TLR2 variants is diagnostic or predictive of susceptibility of Crohn's Disease. In another embodiment, the association of sero-reactivity of ASCA, I2, OmpC, or Cbir to the P631H variant of the TLR2 gene is diagnostic or predictive of susceptibility of Crohn's Disease. In another embodiment, the association of sero-reactivity of ASCA, I2, OmpC, or Cbir to the P631H variant of the TLR2 gene is diagnostic or predictive of susceptibility of Crohn's Disease in Jewish individuals.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of an IL23R variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of an IL23R variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### NOD2: Serologic Analysis and Classification

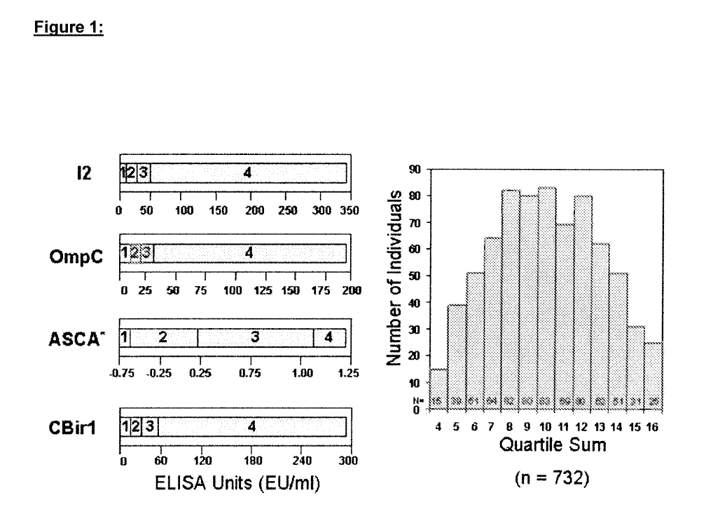

Sera were analyzed for expression of ASCA, anti-I2, anti-OmpC, in a blinded fashion by enzyme-linked immunosorbent assay (ELISA). Antibody levels were determined and results expressed as ELISA units (EU/ml) that are relative to a Cedars-Sinai laboratory (IgA-I2, IgA-OmpC) or a Prometheus Laboratory standard (San Diego, Calif., IgA and IgG ASCA) derived from a pool of patient sera with well-characterized disease found to have reactivity to these antigens. Quantitation of IgG anti-Cbir1 reactivity was expressed in ELISA units derived based on a proportion of reactivity relative to a standardized positive control. As ASCA can be expressed in both an IgA and IgG class, positivity to ASCA was determined if either class of antibody was above the reference range. In determining a quantitative measure of ASCA, the reactivity was first log-transformed and standardized. The higher of two standardized units was then used to determine the quartile of reactivity. With the exception of determining variance (see statistical analysis), the magnitude of reactivity to the other three antigens was not standardized as each is represented by a single class of antibody. The magnitude of the serologic response to each antigen was divided into four equal quartiles in CD patients, unaffected relatives and healthy controls, evaluated as three separate cohorts, to determine quartile sum scores. FIG. 1 shows the patients with the serologic response to each antigen broken down by quartiles and assigned scores of 1-4 on the basis of their designated quartile. By adding individual quartile scores for each microbial antigen, a quartile sum (QS) (range, 4-16) was derived that represents the cumulative semi-quantitative immune response toward all 4 antigens. The quartile ranking reflects the pool of individuals under study (i.e. CD patient or unaffected relative or healthy control) and is not directly comparable between groups.

### Example 2

### NOD2: Genotyping

Three NOD2 variants (R702W, G908R, and 1007fs), were adapted to the TaqMan MGB (Applied Biosystems, Foster City, Calif.) genotyping platform.

### Example 3

### NOD2: Statistical Analysis

The inventors assessed the relationship between carriage of a NOD2, TLR2, TLR4, and TLR9 variant and collective sero-reactivity to microbial antigens both qualitatively and semi-quantitatively. The inventors then determined if any particular NOD2 variant was predominant and examined whether any particular antibody or combinations of antibodies was predominant in determining the relationship between NOD2 variants and sero-reactivity. The contribution of NOD2 to collective sero-reactivity was evaluated by calculating the percent of variance that could be attributed to the presence of NOD2 variants. Finally, the inventors examined whether the presence of a NOD2 variant was related to sero-reactivity to microbial antigens in unaffected relatives of CD patients and healthy controls.

To determine the significance of increasing frequency of carriage of any NOD2 variants with increasing numbers of qualitatively positive antibodies and with increasing quartile sum (range, 4-16), the Cochran-Armitage trend test was performed. To test for differences in the mean quartile sum between those individuals with no NOD2 variant versus those with any variant, the student's t-test was used since the distribution was approximately a normal distribution. One-way ANOVA analysis was done to test the linear trend of mean quartile sum among those with 0, 1, and 2 NOD2 variants. One-way ANOVA analysis was used to test for a difference in sero-reactivity associated with specific NOD2 variants and similarly when comparing mean quartile sum between differing TLR genotypes.

The non-parametric Mann-Whitney test was used to compare the level of seroreactivity between those individuals who carried versus those who did not carry a NOD2 variant for each antibody. To identify whether there is a significant difference in the frequency of carriage of a NOD2 variant among groups within each set with single, double and triple antibody positivity, chi-square analysis was performed.

To determine what proportion of the variation in the sero-reactivity to microbial antigens was attributable to the presence of a NOD2 variant, a coefficient of determination (R2), defined as 1-SS (regression)/SS (total) in ANOVA was used. Sero-reactivity was defined, for this analysis, as the sum of the 4 standardized antibodies, where anti-OmpC=[log(anti-OmpC)−mean(log(anti-OmpC))]/SD(log(anti-OmpC)) and similarly for the other antibodies.

All analyses were performed using SAS computer software (version 8.2; SAS institute, Inc., Cary, N.C., USA, 1999).

### Example 4

### NOD2

The inventors examined serologic and genetic data in 748 Crohn's Disease patients. ASCA and antibodies of I2, OmpC, and Cbir were measured by ELISA. Antibody sums (AS) and overall quartile sums (QS) (ranging from 4-16) of levels for all four antibodies were calculated as previously described (Mow et al Gastro 2004; 126:414). Genotyping (TaqmanMGB) was performed for 3 CD-associated variants of the NOD2 gene, R702W, G908R, and 1007fs.

ASCA was detected in 51%, anti-I2 in 58%, anti-OmpC in 38%, and anti-Cbir1 in 56%. 250 of 748 Crohn's Disease patients (33.4%) had at least one NOD2 variant; 206 (27.5%) having one and 44 (5.9%) having two. NOD2 variants were present at increasing frequency in patients with reactivity to increasing numbers of antigens. Variants were present in those with 0,1, 2, 3, or 4 positive antibodies in 24%, 25%, 36%, 36%, and 46%, respectively (p for trend, 0.0001). NOD2 variants were present at increasing frequency in patients with increasing cumulative quantitative immune response as reflected by individual QS (p for trend, 0.0001). QS were also clustered into four groups by increasing cumulative quantitative immune response (group 1=4-6, group 2=7-9, group 3=10-13, and group 4=14-16). The frequency of having at least NOD2 variant in each of the four groups was 22%, 29%, 35%, and 49% in groups 1, 2, 3, and 4, respectively (p for trend, 0.0001). The mean AS (number of positive antibodies) and QS was higher for patients with at least one NOD2 variant versus those with no variant (2.28±1.21 and 10.70±2.99 vs. 1.90±1.23 and 9.68±2.97, respectively. P, 0.0001).

Individuals with Crohn's disease who have variants of the NOD2 gene as a marker of abnormal innate immunity are more likely to have an increased adaptive immune response to multiple enteric organisms. The data provides a pathophysiologic link to similar findings in rodent mucosal inflammation. This allows disease relevant crossover genetic and functional studies.

### Example 5

### TLR8

The inventors examined a case-control cohort consisting of 763 Crohn's Disease patients, 351 ulcerative colitis patients, and 254 control patients. The patients were genotyped using Illumina technology. SNPs were chosen to tag common Caucasian haplotypes using information from the Innate Immunity PGA.

Both a “risk” and a “protective” TLR8 haplotype were associated with CD in females (risk haplotype (H3): 18% of CD subjects had H3 compared with 8.9% of control subjects; protective haplotype (H2): 59% of CD subjects had H2 compared to 72% of control subjects). No significant association with TLR8 and CD in males was observed. H2 was also associated with UC in females (59% of UC females had H2 compared with 72% of controls, p=0.024) as well as males (32% of UC males had H2 compared with 47% of controls, p=0.009).

Table 4. The odds ratio for CD and UC in females increased progressively as a factor of haplotype combinations from protective to risk:

TABLE 4

| Odds Ratio | H2/H2 | H2/no H3 | Other | H3 positive | P value* |
|---|---|---|---|---|---|
| CD | 0.4 | 0.7 | 1 | 2 | 0.0002 |
| UC | 0.5 | 0.78 | 1 | 2.2 | 0.0032 |
| IBD | 0.43 | 0.7 | 1 | 2.1 | 0.0002 |

(*Mantel-Haenszel)

TLR8 is an X-linked IBD susceptibility gene, with common haplotypes predisposing and protecting. The associations further emphasize the importance of gene variation in innate immunity as genetic determinants, not only of CD, but of UC as well.

### Example 6

### TLR2

The inventors studied if the relationship between variants in innate immune receptors and sero-reactivity to microbial antigens differed in Jewish (J) versus non-Jewish (NJ) patients with CD. Sera from 731 CD patients (282 J, 449 NJ) was tested for ASCA, anti-I2, anti-OmpC, and anti-CBir1 by ELISA while DNA was tested for five TLR2, two TLR4, and two TLR9 variants. The magnitude of responses to microbial antigens was examined according to variant status. Overall quartile sums (QS) (ranging from 4-16) of levels for all four antibodies were calculated as previously described (Mow et al Gastro 2004; 126:414).

There is no association between any TLR4 or 9 variant and sero-reactivity to microbial antigens in Jewish or non-Jewish patients with CD. There is an association between the non-synonymous, non-conservative P631H variant of TLR2 and ASCA positivity in Jewish patients (OR 2.75, p for interaction=0.01). There is an association between the P631H variant of TLR2 and cumulative quantitative response to microbial antigens in Jewish patients with CD. QS were clustered into four groups by increasing cumulative quantitative immune response (group 1=4-6, group 2=7-9, group 3=10-13, and group 4=14-16). The frequency of carriage of the P631H variant of TLR2 increased in parallel with QS cluster in Jewish patients; 2.86%, 3.70%, 7.02%, and 13.46% in groups 1, 2, 3, and 4, respectively (p for trend=0.03). No similar association is found in non-Jewish patients; 7.14%, 10.42%, 6.67%, and 5.45% in groups 1,2,3, and 4, respectively (p for trend=0.40).

Jewish, but not non-Jewish patients with CD who carry the P631H variant of TLR2 have increased sero-reactivity to microbial antigens. The data adds evidence to the paradigm that, in CD, innate immune defects lead to enhanced adaptive immune response to microbial antigens. The differential response to the same genetic variant in two different populations shows a possible gene-gene interaction consistent with the multigenic nature of CD.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

What is claimed is:

1. A method of diagnosing susceptibility to Crohn's Disease in an individual, comprising:
determining the presence or absence of at least one risk variant at the NOD2 locus selected from the group consisting of R702W, G908R and 1007fs, and
determining the presence or absence of at least one risk serological marker,
wherein the presence of at least one risk variant and at least one risk serological marker is diagnostic of susceptibility to Crohn's Disease.

2. The method of claim 1, wherein the presence of three of said risk variants at the NOD2 locus presents a greater susceptibility than the presence of two, one or none of said risk variants at the NOD2 locus, and the presence of two of said risk variants at the NOD2 locus presents a greater susceptibility than the presence of one or none of said risk variants at the NOD2 locus but less than the presence of three risk variants at the NOD2 locus, and the presence of one of said risk variants at the NOD2 locus presents a greater susceptibility than the presence of none of said risk variants at the NOD2 locus but less than the presence of three or two of said risk variants at the NOD2 locus.

3. The method of claim 2, wherein said variant R702W comprises SEQ. ID. NO.: 3.

4. The method of claim 2, wherein said variant G908R comprises SEQ. ID. NO.: 4.

5. The method of claim 2, wherein said variant 1007fs comprises SEQ. ID. NO. 5.

6. The method of claim 1, wherein said risk serological markers are selected from the group consisting of ASCA, I2, OmpC and Cbir.

7. The method of claim 6, wherein the presence of four of said risk serological markers presents a greater susceptibility than the presence of three or two or one or none of said risk serological markers, and the presence of three of said risk serological markers presents a greater susceptibility than the presence of two or one or none of said risk serological markers but less than the presence of four risk serological markers, and the presence of two of said risk serological markers presents a greater susceptibility than the presence of one or none of said risk serological markers but less than the presence of four or three risk serological markers, and the presence of one of said risk serological markers presents a greater susceptibility than the presence of none of said risk serological markers but less than the presence of four or three or two of said risk serological markers.

8. The method of claim 1, further comprising the step of determining the presence or absence of one or more risk haplotypes at the TLR8 locus, wherein the presence of one or more risk haplotypes at the TLR8 locus is diagnostic of susceptibility to Crohn's Disease.

9. The method of claim 1, further comprising the step of determining the presence or absence of one or more risk haplotypes at the TLR2 locus, wherein the presence of one or more risk haplotypes at the TLR2 locus is diagnostic of susceptibility to Crohn's Disease.

10. A method of diagnosing susceptibility to Crohn's Disease in an individual comprising:
determining the presence or absence of one or more risk haplotypes at the TLR8 locus in the individual,
wherein the presence of one or more risk haplotypes is diagnostic of susceptibility to Crohn's Disease.

11. The method of claim 10, wherein said individual is a female.

12. The method of claim 10, wherein one of said one or more risk haplotypes is H3.

13. The method of claim 10, wherein the one or more risk haplotypes comprise one or more variant alleles selected from SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and SEQ. ID. NO.: 16.

14. A method of determining a low probability relative to a healthy individual of developing Crohn's Disease and/or ulcerative colitis in an individual, said method comprising:
determining the presence or absence of one or more protective haplotypes at the TLR8 locus in the individual,
wherein the presence of one or more of said protective haplotypes is diagnostic of a low probability relative to a healthy individual of developing Crohn's Disease and/or ulcerative colitis.

15. The method of claim 14, wherein said individual is a female.

16. The method of claim 14, wherein one of said one or more protective haplotypes is H2.

17. The method of claim 14, wherein the one or more protective haplotypes comprise one or more variant alleles selected from SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and SEQ. ID. NO.: 16.

18. A method of diagnosing susceptibility to Crohn's Disease in an individual comprising:
determining the presence or absence of one or more risk variants at the TLR2 locus in the individual,
wherein the presence of one or more risk variants is diagnostic of susceptibility to Crohn's Disease.

19. The method of claim 18, wherein said individual is Jewish.

20. The method of claim 18, wherein one of the one or more risk variants is P631H at the TLR2 locus.

21. The method of claim 20, wherein P631H comprises SEQ. ID. NO.: 18.
